(19) United States
(12) Patent Application Publication
(10) Pub. No.: US 2025/0296903 A1
(43) Pub. Date: Sep. 25, 2025

(54) SYSTEMS AND METHODS FOR PRODUCING SURFACTANTS AND DETERGENT COMPOSITIONS USING LIGHTLY BRANCHED ALCOHOLS

(71) Applicant: ExxonMobil Technology and Engineering Company, Spring, TX (US)

(72) Inventors: Chunzhao Li, Spring, TX (US); Travis A. Reine, Seabrook, TX (US); Geert Steenbeke, Lovendegem (BE); Shiwen Li, Quaregnon (BE); Roxana Perez Velez, Leuven (BE); Ouissam Abbas, Mechelen (BE); Francis Gillis-Dhamers, Moerbeke-Waas (BE)

(21) Appl. No.: 19/082,346

(22) Filed: Mar. 18, 2025

Related U.S. Application Data
(60) Provisional application No. 63/567,231, filed on Mar. 19, 2024.
(60) Provisional application No. 63/772,986, filed on Mar. 17, 2025.

(51) Int. Cl. C07C 29/48; C11D 1/12; C11D 1/66

(52) U.S. Cl. CPC C07C 29/48; C11D 1/12; C11D 1/66; C11D 2111/12

(57) ABSTRACT

Provided herein is a method for generating a lightly branched C₁₃ oxo alcohol and utilizing it to produce a surfactant. The method includes contacting butylene feed stream and an optional propylene feed stream in the presence of a catalyst to produce lightly branched olefins. The lightly branched olefins are hydroformylated in the presence of a catalyst to produce C₁₃ oxo alcohols. Further, the method includes contacting the lightly branched olefin feed stream with surfactant precursors in the presence of a catalyst, the contacting taking place under conditions to catalytically react the surfactant precursor and C₁₃ branched oxo alcohol into a surfactant, making these surfactants particularly suitable for surfactant-based applications.

### CROSS-REFERENCE TO RELATED APPLICATIONS

This application claims priority to and the benefit of U.S. Provisional Application No. 63/567,231, filed on Mar. 19, 2024, and U.S. Provisional Application No. 63/772,986, filed on Mar. 17, 2025, the disclosures of which are incorporated herein by their entireties.

### FIELD

This disclosure relates to a composition of matter and processes for producing the composition of matter including surfactants and detergent compositions exhibiting effective stain removal properties. In particular, the processes to produce surfactants may include using a feed stream having lightly branched C₁₃ oxo alcohols (LBAs) with surfactant precursors. In general, the lightly branched C₁₃ oxo alcohols (LBAs) described herein exhibit limited branching, first branch distribution, and viscosity properties. Accordingly, the composition of matter and processes of this disclosure are especially useful for surfactant-based applications.

### BACKGROUND

Surfactants are amphiphilic compounds that decrease the surface tension of two compositions at an interface. The molecular structure of most commonly found surfactants typically consists of a combination of properties-a hydrophilic component and a hydrophobic component. The differences in polarity between hydrophilic component and the hydrophobic component aid the solubilizing of insoluble or slightly chemicals (e.g., that would otherwise not solubilize or only partially solubilize in the absence of heat and/or surfactant). Additional surfactants may be used to decrease the viscosity of a fluid phase or enhancing foaming properties of a fluid. Accordingly, surfactants may be implemented into a variety of consumer and industrial products, including, but not limited to, detergents, emulsifiers, cosmetics, pharmaceuticals, and dispersants.

Conventional surfactants may exhibit poor performance and solubility when employed in cold water conditions. Ostensibly, branched surfactants have been found to be effective detergents in cold water, yet many of the conventional branched surfactants exhibit poor cleaning performance and stain removal properties at low temperatures (e.g., less than 30° C.). Therefore, there is currently a need for surfactants that exhibit good cleaning performance and stain removal properties in cold water at temperatures less than about 30° C.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below. As discussed above, it is desirable to generate surfactants having good cleaning performance and stain removal properties in cold water. Certain conventional surfactants used in certain industrial processes may exhibit poor solubility or detergency performance in cold water that prevent the surfactants from removing stains. Accordingly, it would be advantageous to produce (e.g., generate) surfactants that have minimal branching, while also exhibiting good cleaning performance and stain removal properties. It is presently recognized surfactants having limited branching and first branch distribution may provide satisfactory surfactant-based applications in which conventional surfactants may be unsuitable due to poor cleaning performance and stain removal properties in cold water. Accordingly, it is presently recognized that it may be advantageous to develop techniques that produce surfactants having limited branching and first branch distribution.

This disclosure relates to techniques for generating lightly branched C₁₃ oxo alcohols (LBAs) that have limited (e.g., light) branching (e.g., low branching index values) and a first branch distribution. Further, the disclosure relates to generating surfactants based on the LBAs that have limited branching and a first branch distribution. In general, the techniques discussed herein include providing a mixture comprising butene and, optionally a small amount of propylene in the presence of a catalyst to generate lightly branched C₁₂ olefins (LBOs). Further, the LBOs are hydroformylated in the presence of a hydroformylation catalyst to produce a lightly branched C₁₃ oxo alcohol (LBA) composition. In particular, the LBA compositions described herein may exhibit branching index (BI) values between about 1.3 and 1.7 (e.g., about 1.3, 1.4, 1.5, 1.6, or 1.7) and an average carbon number between about 12.5 and 13.5 (e.g., about 12.5, 12.75, 13, 13.25, or 13.5). Further, the conditions described herein may provide an LBA composition including a defined distribution of the first branch position, wherein the first branch position is determined relative to the hydroxyl group based on the overall LBA composition (e.g., about 10 to about 20% first branch at position two, about 20 to about 40% first branch at position three, about 5 to about 15% first branch at position four, and about 35 to about 55% first branch at position five and beyond (5 & 5+), as characterized by C₁₃ NMR.

In some embodiments, the disclosed techniques may include generating surfactants using the disclosed C₁₃ oxo alcohol. Accordingly, a reaction can be performed between the disclosed C₁₃ oxo alcohol and surfactant precursors (e.g., ethylene oxide, sulfur trioxide, or chlorosulfonic acid, or a combination) in the presence of an optional catalyst to produce surfactants (e.g., alcohol derived sulfate or an alcohol derived ethoxylate), respectively. The surfactants made from the disclosed LBA exhibit a surprising stain removal property improvement in cold water relative to conventional branched C₁₃ alcohols.

These and other features and attributes of the present disclosure and their advantageous applications and/or uses will be apparent from the detailed description which follows.

### BRIEF DESCRIPTION OF THE DRAWINGS

To assist those of ordinary skill in the relevant art in making and using the subject matter hereof, reference is made to the appended drawings, wherein:

FIG. 1 is a flow diagram of a method for producing branched C₁₃ oxo alcohols, in accordance with the present disclosure;

FIG. 2 illustrates an example molecular structure of a branched C₁₃ oxo alcohol, in accordance with the present disclosure;

FIG. 3 is a flow diagram of a method for producing surfactants, in accordance with the present disclosure;

FIG. 4 is a block diagram of a system that may determine branching properties and generate outputs based on the branching properties, in accordance with the present disclosure;

FIG. 5 is a flow diagram of a method for generating a correlation linear regression model including Fourier transform infrared-attenuated total reflectance (FTIR-ATR) and nuclear magnetic resonance (NMR) data, in accordance with the present disclosure;

FIG. 6 is an example FTIR-ATR spectrum of branched alcohol samples in the 2700 cm⁻¹ to 3100 cm⁻¹ region, in accordance with the present disclosure;

FIG. 7 is an example FTIR-ATR spectrum of branched alcohol samples in the 1330 cm⁻¹ to 1480 cm⁻¹ region, in accordance with the present disclosure;

FIG. 8 is a graph of a correlation model including FTIR-ATR and ¹H NMR data, in accordance with the present disclosure;

FIG. 9 is an example FTIR-ATR spectrum of branched alcohols before Fourier self-deconvolution in the in the 936 cm⁻¹ to 1140 cm⁻¹ region, in accordance with the present disclosure;

FIG. 10 is the example FTIR-ATR spectrum of branched alcohols after Fourier self-deconvolution in the in the 936 cm⁻¹ to 1140 cm⁻¹ region, in accordance with the present disclosure; and

FIG. 11 is a graph of a correlation model including FTIR-ATR and ¹³C NMR data, in accordance with the present disclosure.

### DETAILED DESCRIPTION

One or more specific embodiments of the present disclosure will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements. All numerical values within the detailed description herein are modified by “about” the indicated value, and take into account experimental error and variations that would be expected by a person having ordinary skill in the art. For example, “about” or “approximately” may refer to ±0.5%, ±1%, ±2, ±5%, ±10%, or ±15%.

As used herein, a “carbon number” refers to the number of carbon atoms in a hydrocarbon. Likewise, a “Cx” hydrocarbon is one having x carbon atoms (i.e., carbon number of x), and a “Cx-Cy” or “Cx-y” hydrocarbon is one having from x to y carbon atoms.

The term “alkane” refers to non-aromatic saturated hydrocarbons with the general formula CₙH(2n+2), where n is 1 or greater. An alkane may be straight chained or branched. Examples of alkanes include methane, ethane, propane, butane, pentane, hexane, heptane and octane. “Alkane” is intended to embrace all structural isomeric forms of an alkane. For example, butane encompasses n-butane and isobutane; pentane encompasses n-pentane, isopentane and neopentane.

The term “olefin,” and “alkene,” are used interchangeably to refer to a branched or unbranched unsaturated hydrocarbon having one or more carbon-carbon double bonds. A simple olefin comprises the general formula CnH(2n), where n is 2 or greater. Examples of olefins include ethylene, propylene, butylene, pentene, hexene and heptene. “Olefin” is intended to embrace all structural isomeric forms of an olefin. For example, butylene encompasses but-1-ene, (Z)-but-2-ene, etc.

The term “reactor” refers to any vessel(s) in which a chemical reaction occurs. Reactor includes both distinct reactors, as well as reaction zones within a single reactor apparatus and, as applicable, reactions zones across multiple reactors. For example, a single reactor may have multiple reaction zones.

The terms “branched”, “lightly branched”, and “branched hydrocarbon” refer to a hydrocarbon or hydrocarbyl group having a linear main carbon chain in which a hydrocarbyl side chain extends from the linear main carbon chain. The term “unbranched” refers to a straight-chain hydrocarbon or hydrocarbyl group without side chain groups extending therefrom. More particularly, “lightly branched” refers to hydrocarbons having branches (e.g., monobranched, dibranched, tribranched). It should be noted that the NMR techniques discussed herein may determine composition based off the position of the chain.

The term “lightly branched olefin (LBO)” refers to an alkenic hydrocarbon bearing a carbon-carbon double bond within the main carbon chain. While side chain branches are present in an LBO, the branching is minimal (e.g., one side branch, two side branches, or three side branches with an average branch of 1.5 branch per molecule) in a given LBO sample.

The term “first branch” refers to the first hydrocarbyl side chain closest to the hydroxyl group extending from the linear main carbon chain. The term “second branch” refers to the second hydrocarbyl side chain farthest from the hydroxyl group extending from the linear main carbon chain that is not the first branch.

The term “branch position” refers to the position of the first branch along the main carbon chain, wherein the first CH2 group on the main chain bonded to the hydroxyl group is referred to as “position one.” Accordingly, the “branch position” of the first branch may be position one, position two (e.g., second position, 2^{nd} position), position three, position four, position 5, or position 5 and beyond (5 & 5+).

In general, a “branching index” be determined using proton NMR based on the peak integral from the ppm range corresponding to methylene protons adjacent to the hydroxyl group, remaining aliphatic and hydroxyl protons and methyl protons (CH3). A “branch position” may be determined using Carbon-13 NMR (¹³C NMR) based on the peak integral from the ppm range corresponding to the position of the first branch. It should be noted that the position of the first branch is distinguishable up to position 4. Branched C₁₃ oxo alcohols having a first branch at position 5 & 5+ are not distinguishable. Similarly, the position of the second branch is indistinguishable due to complexities arising within ¹³C NMR. It is to be understood that other techniques such as FT-IR, NIR, Raman, HPLC, GC, GC-MS might be applicable for the determination of one or more of the properties of the disclosed invention.

The term “surfactant” refers to amphiphilic compounds comprising a hydrophilic portion and a hydrophobic portion that tend to lower the surface tension at an interface between two components. Surfactants are amphiphilic compounds comprising a hydrophilic portion and a hydrophobic portion that tend to lower the surface tension at an interface between two components. As such, surfactants may be used in a wide range of applications, which may include, for example, promoting solubility of an otherwise sparingly soluble material, lowering viscosity of a fluid phase, and promoting foaming of a fluid. Surfactants may be found in a wide range of consumer and industrial products including, for example, soaps, detergents, cosmetics, pharmaceuticals, and dispersants. Ionic functional groups that may be present in the hydrophilic portion of surfactants include, for example, sulfonates, sulfates, carboxylates, phosphates, quaternary ammonium groups, and the like. Non-ionic hydrophilic portions may include functional groups or moieties bearing one or more heteroatoms that are capable of receiving hydrogen bonds, such as polyethers (e.g., ethoxylates). Zwitterionic hydrophilic portions may include moieties such as betaines, sultaines, and related phospholipid compounds. It should be noted that “surfactants” or “amphiphiles” may be used interchangeably herein.

The term or phrase “detergent composition” or refers to compositions and formulations designed for cleaning and removing stains from soiled materials. Examples of such compositions include, but are not limited to, laundry cleaning, fabric softeners, laundry additives, dry cleaning materials, laundry rinse additives, dish washing, hard surfaces, detergents in other materials. The detergent compositions described herein may be formulated into various forms such as liquids, powders, gels, paste, bars, tablets, pouches, doses, etc. For example, a detergent composition 74 may include the disclosed LBA 32 in combination with one or more additives, such as stabilizers, alkali ingredients, and additional surfactants (e.g., second surfactant, third surfactant).

As referred to herein, “substantially free of” or “substantially free from” or “substantially” refers to either the complete absence of a component or includes a minimal amount of the component, such as an impurity or unintended byproduct of another ingredient. For example, a composition that is “substantially free” of/from a component may refer to a composition that includes less than about 0.5%, 0.25%, 0.1%, 0.05%, or 0.01%, or even 0%, by weight of the composition, of the component.

The term “SRI” refers to stain removal index or soil removal index.

It should be noted that the disclosed techniques describe C₁₃ oxo alcohol to produce surfactants. The C₁₃ oxo alcohol may also be utilized to produce surfactants including additional nonionic surfactants, anionic surfactants, cationic surfactants. The disclosed C₁₃ oxo alcohol could also be used to produce esters, and acrylates, accordingly.

NMR analyses such as quantitative ¹H NMR and ¹³C NMR spectroscopy methods have been applied to determine the branching index and branching at the 2nd position.

Fourier transform infrared (FTIR)-attenuated total reflectance (ATR) spectroscopy of alcohols was performed to determining branching and branching at 2nd position. A non-specific limiting example of FTIR-ATR may include acquiring FTIR-ATR data on an infrared spectrometer using a smartITR device equipped with a ZnSe crystal diamond coated. Spectra were recorded at room temperature (about 21° C.). Spectra are obtained by providing a drop of the alcohol sample on the ATR crystal. Background was collected on air. Spectra were recorded in triplicates at 4 cm⁻¹ of spectral resolution and a scan co-adding equal to 64. Spectra cover the range from 4000 to 600 cm⁻¹

Spectral data treatment for FTIR spectra-Functional and structural indexes are calculated from the ratio of bands area (e.g., integrated from valley to valley). Areas are integrated and denoted “Areaᵥ” where ν is the frequency corresponding to the band maximum. Ratios have also been calculated based on signal intensity. It was designated “lᵥ” where ν is the frequency corresponding to the peak maximum height. It should be noted that these FTIR-ATR acquisition parameters are meant to be exemplary and may be modified accordingly.

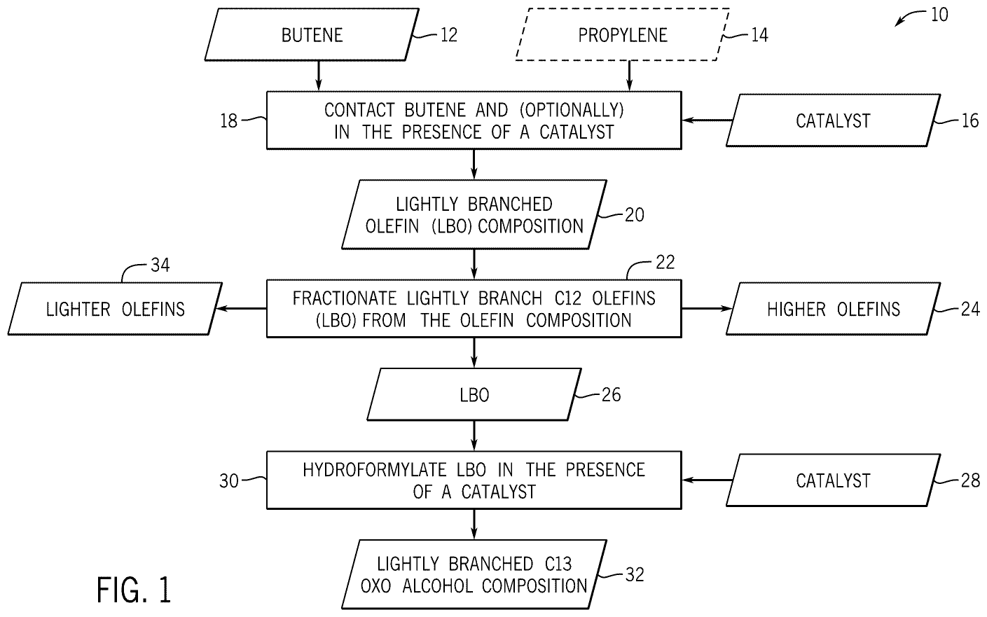

Reference is now made to the embodiments illustrated in FIGS. 1-3, wherein like numerals are used to designate like parts throughout.

### Lightly Branched Alcohols

FIG. 1 illustrates a flow diagram of method 10 for producing lightly branched C₁₃ oxo alcohols (LBAs) in accordance with certain embodiments of the present disclosure. As shown, the method 10 includes providing butene 12 (e.g., a butene feedstock, wherein the butene feedstock may include isomers of butene (e.g., 1-butene, 2-butene, isobutylene) and, optionally, propylene 14 (e.g., a propylene feedstock) in the presence of a catalyst 16 to produce an LBO composition 20.

Referring to the method 10, at block 18, butene 12 and the optional propylene 14 are contacted in the presence of a catalyst 16 such as certain conditions described in U.S. Pat. No. 11,905,227 B2, U.S. Pat. No. 11,312,669 B2, WO2022233875A1, WO2022233876A1, which are incorporated herein by reference. In some embodiments contacting butene 12 and, optionally, propylene 14 in the presence of the catalyst 16 may include providing a flow of a feedstock (e.g., butene feed flow rate and the optional propylene feed flow rate) including the butene 12 and the optional propylene 14 over a solid support formed of the catalyst 16 into a reactor. For example, the catalyst 16 may be stored or otherwise contained in a reaction vessel, and the feedstock including butene 12 and propylene 14 may be provided, flowed, or otherwise directed into the reaction vessel including the catalyst 16. In some embodiments, the reactor may be a single fixed bed reactor or preferably a multi-tubular reactor.

Solid acid catalysts suitable for producing olefin oligomers having an average branching index of about 2.2 or less, particularly for C₁₂ olefin oligomers having an average branching index of about 2.2 or less, such as an average branching index of about 1.0 to about 1.9 may include, for example, zeolite catalysts having an MTT or TON framework, including unmodified zeolite catalysts having these frameworks. Suitable examples may include, for instance, ZSM-22, ZSM-23, ZSM-57, and SAPO-11. Such solid acid catalysts and other zeolite catalysts may be modified by steaming, modified with an organic acid, modified with a transition metal, modified with coke, impregnated with NiO, or any combination thereof. Suitable modification conditions are described further below. Although already suitable for producing an average branching index of about 2.2 or less, such modifications to these zeolite catalysts may further improve selectivity and/or decrease the average branching index, as explained further below. Such solid acid catalysts may afford selectivity for forming C₁₀-C₁₃ olefin oligomers when exposed to suitable oligomerization reaction conditions.

Suitable zeolite catalysts, such as ZSM-23, can be prepared from extrudates (about 1 wt. % to about 90 wt. % binder and about 10 wt. % to about 99 wt. % zeolite) or from zeolite crystal seeds. Examples of suitable binders may include silica, alumina, zirconia, titania, silica-alumina, metal oxides, the like, and mixtures thereof. Particular zeolite catalysts may be crystalline and have an aspect ratio of about 1 to about 5, alternatively about 2 to about 4, with a width of less than about 0.1 microns and a length of less than about 0.3 microns. Prior to use, the zeolite catalysts may be calcined in air at about 425° C. to about 650° C. for about 1 hour to overnight.

Particular zeolite catalyst examples may include, for example, a Si/Al ZSM-23 catalyst having no amine treatment and a Si: A12 molar ratio of about 20 to about 60, or about 25 to about 55, or about 30 to about 50, or about 30 to 45. Si/Al ZSM-23 catalysts may be prepared as described in U.S. Pat. Nos. 4,076,842 and 5,332,566, each of which is incorporated herein by reference. Alternatively, the zeolite catalyst may be a Si/Al/Ti ZSM-23 catalyst having no amine treatment and a Si: A12 molar ratio of about 20 to about 60, or about 25 to about 55, or about 30 to about 50 and a Ti: Al molar ratio of about 0.1 to about 3, or about 0.2 to about 2, or about 0.3 to about 1. Si/Al/Ti ZSM-23 catalysts may be prepared as described in the foregoing U.S. patents. A combination of the two ZSM-23 catalyst types may be used. In still other examples, the zeolite catalyst may have a Si: A12 molar ratio of about 30:1 to about 200:1 and comprise about 0.1 wt. % to about 5 wt. % transition metal and about 0.1 wt. % to about 3.3 wt. % framework Al—O. In general, preparation of the zeolite catalysts described herein may be prepared as described in WO2022233879A1, which is incorporated herein by reference.

Oligomerization may be carried out in a fixed bed reactor, a packed bed reactor, a tubular reactor, a fluidized bed reactor, a slurry reactor, a continuous catalyst regeneration reactor, or any combination thereof. Suitable oligomerization reaction conditions may include a reaction temperature of about 80° C. to about 350° C., or about 90° C. to about 350° C., or about 150° C. to about 350° C., or about 170° C. to about 310° C. Oligomerization may take place at a pressure ranging from about 50 bar to about 300 bar, or about 60 bar to about 150 bar, or about 70 bar to about 120 bar.

Oligomerization may be carried out at a WHSV ranging from about 2 hr⁻¹ to 70 hr⁻¹, or about 5 hr⁻¹ to about 30 hr⁻¹, or about 5 hr⁻¹ to about 10 hr⁻¹, or about 10 hr⁻¹ to about 15 hr, or about 15 hr⁻¹ to about 20 hr⁻¹, or about 20 hr⁻¹ to 30 hr⁻¹. Surprisingly, certain zeolite catalysts, particularly those having an MTT framework, such as ZSM-23 or modified ZSM-23, may promote formation of C10-C13 olefin oligomers with relatively good selectivity, while affording a branching index for at least C12 olefins of about 1.7 or less particularly about 1.1 to about 1.7.

More specifically, the reaction vessel may include a solid acid component that promotes formation of lightly branched olefins having a range of methyl group and double bond positions.

Referring to the method 10, at block 22, the resulting LBO composition 20 is separated (e.g., fractionated) to produce higher olefins 24 (e.g., olefins heavier than C₁₂, such as C₁₆, C₂₀, C₂₄, etc.), lighter olefins 34 (e.g., olefins lighter than C₁₂, such as C₄ feed, C₈), and lightly branched olefins (LBO) 26 (e.g., lightly branched C₁₂ olefins including one or more of linear dodecenes, mono-alkyl (e.g., mono-methyl, mono-ethyl, mono-n-propyl, mono-i-propyl) branched isododecenes, dibranched isododecenes, multi-branched isododecenes, and trace amounts of dienes and/or cyclic alkanes). In general, fractionating may include providing, flowing, or otherwise directing for fractionation for separation using suitable techniques, such as distillation and other techniques understood by a person of ordinary skill in the art. Accordingly, the LBOs 26 exhibit a branching index in the range of 1.0 to 1.9, more preferably between 1.1 to 1.8, 1.2 to 1.7, 1.3 to 1.6.

Referring to the method 10, at block 30, LBOs 26 are contacted in the presence of a catalyst 28 that causes the LBOs 26 to undergo hydroformylation (i.e., a reaction in the presence of carbon monoxide in hydrogen with a catalyst), thereby producing the LBA 32 (i.e., a primary alcohol). As described here, the LBAs may include advantageous properties, or combinations of properties, such as branching index, viscosity, for use as a feedstock for producing surfactants.

The catalyst 28 may include a suitable transition metal complex (e.g., cobalt-based catalysts, ruthenium-based catalysts, iridium-based catalyst, etc.) or suitable compound that facilitates hydroformylation. In a similar manner as described with respect to block 12, contacting the LBO 26 in the presence of the catalyst 28 and/or catalyst platform 28 may include providing a flow of a feedstock (e.g., LBO feed flow rate) over the catalyst 28 and/or catalyst platform 28. For example, the catalyst 28 and/or catalyst platform 28 may be stored or otherwise contained in a reaction vessel, and the feedstock including LBO 26 may be provided, flowed, or otherwise directed into the reaction vessel including the catalyst 28 and/or catalyst platform 28. In another embodiment, referring to block 30, LBO 26 may be contacted in a reactor with a homogeneous catalyst 28. For example, the catalyst 28 may be dissolved in a reaction medium in the reactor.

During the process of hydroformylation, LBO 26 is initially converted into an aldehyde. Subsequently, the aldehyde is reduced via hydrogenation, producing lightly branched C₁₃ oxo alcohol (LBA) composition 32. In general, the aldehyde can undergo hydrogenation during the hydroformylation process. In another embodiment, the aldehyde may be provided, flowed or otherwise directed to an additional catalyst (i.e., different than the catalyst 28) for hydrogenation after hydroformylation. The catalyst may be a heterogenous catalyst. The LBAs 32 may include advantageous properties, or combinations of properties, such as branching index, viscosity, for use as a feedstock for producing surfactants, or overall weight percent of isomer composition.

Suitable catalysts for promoting hydroformylation of one or more lightly branched olefins may include a metal carbonyl complex, such as a carbon monoxide complex of a transition metal of Groups 8-10 of the Periodic Table. Of the Group 9 metals, cobalt and rhodium are best known for their hydroformylation activity, but other suitable metals in Groups 8-10 may include palladium, iridium, ruthenium and platinum. By way of nonlimiting example, suitable catalysts may include HRh(CO)(PR3)3, HRh(CO)2(PR3), HRh(CO)[P(OR)3]3, Rh(CH₃COCH₂COCH₃)(CO)2, Rh6(CO)16, [Rh(norbornadiene)(PPh3)2+[PF6]−, [Rh(C)3(PPh3)2]+[BPh4]−, RhCl(CO)(PEt3)2, [RhCl(cyclooctadiene)]2, [Rh(CO)3(PR3)2]+BPh4−, [Rh(CO)3(PR3)2]+PF6−, HCo(CO)4, Ru3(CO)12, [RuH(CO)(acetonitrile)2(PPh3)3+[BF4]−, PtCl2 (cyclooctadiene), [Ir(CO)3 (PPh3)]+[PF6]−, or [HPt(PEt3)3]+[PF6]−. Other suitable catalysts may include, for example, HCo(CO)4, Co2(CO)8, HCo(CO)3(POR)3 (R=alkyl or aryl), HCo(CO)3(PR3) (R=alkyl or aryl), and Co(II)X2 (X=anionic ligand, such as carboxylate, sulfate, halide, alkoxide, amide, and the like). Particularly suitable cobalt hydroformylation catalysts may include unmodified HCo(CO)4 or Co2(CO)8. Inorganic salts and catalyst precursors, such as Rh2O3, Pd(NO3)2 and Rh(NO3)3, may be used, as may halides such as, for example, RhCl3·3H2O. In exemplary embodiments, a nickel catalyst in the presence of dimethylamine may be used.

Olefin oligomers not undergoing hydroformylation may undergo subsequent reduction into paraffins once the hydroformylation reaction product is converted into a primary alcohol. Paraffins may be separated from the primary alcohols following reduction or maintained therewith.

Reducing may comprise hydrogenating the hydroformylation reaction product in particular embodiments of the present disclosure. Hydrogenation may comprise exposing the hydroformylation reaction product to hydrogen and a hydrogenation catalyst (i.e., catalytic hydrogenation conditions using a catalyst comprising Fe, Co, Ni, Ru, Rh, Cr, Mo, Pd, Os, Ir, or Pt, preferably supported on an inorganic substrate, and a hydrogen partial pressure of, for example, about 5 MPa to about 20 MPa, and a reaction temperature up to about 180° C.). Catalytic hydrogenation may remove any residual carbon-carbon unsaturation present in the hydroformylation reaction product, as well as reduce at least a portion of the aldehyde groups into primary alcohols. Hydride reduction, either conducted alone or in combination with catalytic hydrogenation, may complete the reduction of the aldehyde moieties into a primary alcohol moiety. In an example process configuration, reduction may comprise exposing the hydroformylation reaction product to catalytic hydrogenation to produce a reduced hydroformylation reaction product.

Solvents or diluents are not necessary when conducting the hydroformylation reaction according to the disclosure herein, but may optionally be present in any amount. When used, suitable solvents or diluents may include, but are not limited to, alkane solvents, polar protic solvents, polar aprotic solvents, chlorinated solvents and aromatic solvents. In a particular example, up to about 10 wt. % water may be added to control byproduct formation under the hydroformylation reaction conditions. Without being bound by theory or mechanism, water may hinder the formation of aldol condensates and other heavy reaction products.

Several non-limiting examples of the composition of the LBAs 32 are described below. In general, the compositions described below describe the properties of the LBAs 32.

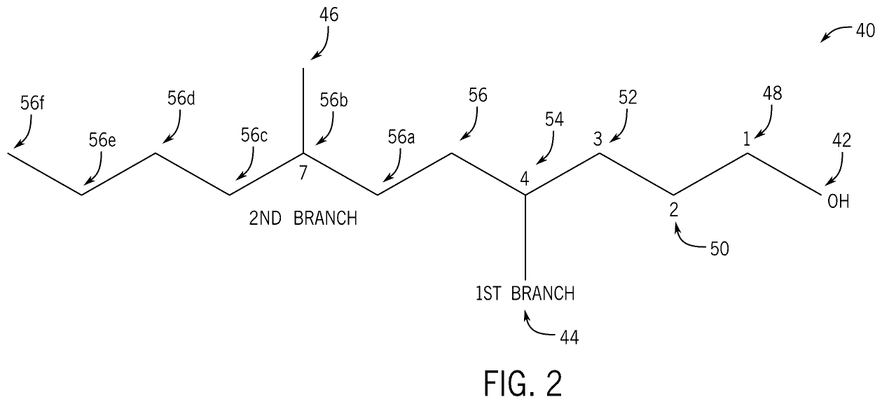

By way of example, FIG. 2 illustrates a molecular structure 40 of the LBA 32, in accordance with the embodiments of the present disclosure. Referring to the method 10, at block 30 of FIG. 1, compositional information regarding the LBA 32 is obtained using C¹³ NMR. The resulting LBA 32 exhibits two branches, a first branch 44 and a second branch 46. It should be noted that the branch (e.g., hydrocarbyl groups) may be methyl groups, ethyl groups, propyl groups, etc. As described herein, the first branch refers to the branch closest to the hydroxyl group 42 on the main carbon chain of the LBA 32. In general, the position of the first branch 44 may be at position one 48, position two 50, position three 52, or position four 54. The first branch may also be at positions beyond position five 56, such as position six 56a, position seven 56b, position eight 56c, position nine 56d, position 10 56e, or position 11 56f. As referred to herein, position five and beyond (e.g., 5&5+) may include position five 56, position six 56a, position seven 56b, position eight 56c, position nine 56d, position 10 56e, or position 11 56f, and may be collectively referred to as position 56. It should be noted that the position of the first branch 44 may be distinguishable in C¹³ NMR up to position 4. As such, LBAs 32 having a first branch 44 at position five 56 and beyond are not distinguishable. Similarly, the position of the second branch is indistinguishable due to complexities arising within ¹³C NMR. It should be noted that the position of the second branch 46 as illustrated in FIG. 2 is an example and can exhibit positions including position 5 and beyond of the LBA 32. As such, it is presently recognized that the techniques described herein include advantages such as determining the position of the first branch within the LBAs 32. In general, the LBAs 32 exhibit an average number of carbons between about 12.5 and about 13.5. For example, the average number of carbons may range between 12.7 and 13.3, 12.9 and 13.1, about 12.6, about 12.8, about 13.0, about 13.2, or about 13.4. Further, the LBAs 32 exhibit a branching index between about 1.0 and about 1.9. For example, the branching index may range between about 1.3 and about 1.7, or 1.4 and 1.6. The disclosed LBAs 32 exhibit kinematic viscosity at 20° C. between 30 to 40 mm²/s, or 32 to 38 mm²/s, or 34 to 36 mm²/s. The disclosed LBAs 32 are also defined by a specific range of first branch position distribution (position 2, 3, 4, 5 & 5+) based on the C13 NMR analysis of the first branch carbons. For example, % distribution of LBAs 32 exhibiting a first branch at position two may range between about 10 to about 20%, a first branch at position three may range between about 20 to about 40%, a first branch at position three may range between about 20 to about 40%, a first branch at position four may range between about 5 to about 15%, and a first branch at position 5 & 5+ 56 may range between about 35% to about 55%.

### Surfactant and Detergent Compositions

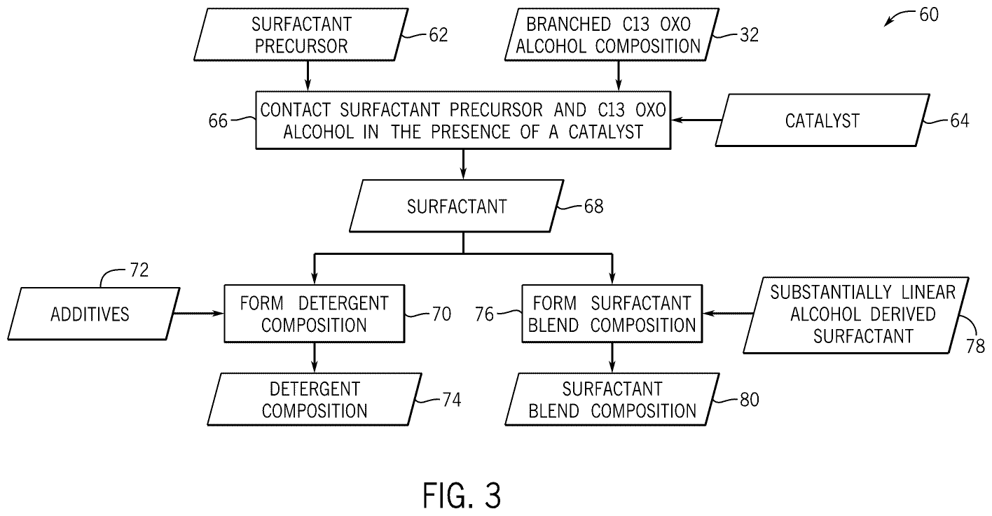

With the foregoing in mind, FIG. 3 illustrates a flow diagram of a method 60 for producing surfactants 68 based on LBAs 32 and surfactant precursor 62, in accordance with certain embodiments of the present disclosure. As shown, the method 60 includes, at block 66, contacting surfactant precursor 62 and the LBAs 32 in the presence of an optional catalyst 64. For example, block 66 may include performing an ethoxylation reaction using the disclosed LBAs 32 and the surfactant precursor 62 (e.g., ethylene oxide, propylene oxide), producing surfactant 68 (e.g., alcohol alkoxylates (e.g., alcohol derived ethoxylates)). In a further example, block 66 may include performing a sulfation reaction using the disclosed LBAs 32 and the surfactant precursor 62 (e.g., sulfur trioxide or chlorosulfonic acid), producing surfactant 68 (e.g., alcohol derived sulfates). It should be noted that alcohol derived ethoxylates may have similar structural features to the alcohol derived sulfates except for their non-ionic hydrophilic group. As described herein, the surfactants 68 (e.g., alcohol derived ethoxylates, alcohol ether sulfates, alcohol derived sulfates) may include advantageous properties, or combinations of properties, such as low BI values (e.g., minimal branching) and stain removal performance.

In some embodiments, contacting LBA 32 and surfactant precursor 62 in the presence of the optional catalyst 64 may include providing a flow of a feedstock (e.g., LBA feed flow rate and surfactant precursor feed flow rate) including the LBA 32 and surfactant precursor 62 (e.g., alcohol derived ethoxylate precursors (e.g., ethylene oxide) or alcohol derived sulfate precursors (e.g., sulfur trioxide, chlorosulfonic acid)) in the presence of the optional catalyst 64. For example, the optional catalyst 64 may be stored or otherwise contained in a reaction vessel, and the feedstock including LBA 32 and surfactant precursor 62 may be provided, flowed, or otherwise directed into the reaction vessel including the optional catalyst 64. The surfactant 68 (e.g., alcohol derived ethoxylate, alcohol ether sulfates, or alcohol derived sulfate) is formed through a reaction between a surfactant precursor 62 and the LBA 32. Advantageously, the resulting surfactant 68 can be utilized as detergent compositions due to its low BI values (e.g., minimal branching) and stain removal performance.

Several non-limiting examples of the composition of the surfactant 68 are described below. In general, the compositions described below describe the properties of the surfactant 68.

In some embodiments, the LBAs 32 may be converted into anionic surfactants. A description of an example techniques for converting alcohols into anionic surfactants, such as alcohol derived sulfates, is described in “Anionic Surfactants-Organic Chemistry”, Volume 56 of the Surfactant Science Series, Marcel Dekker, New York. 1996. It should be noted that such techniques may be applied to the disclosed LBAs 32.

Accordingly, surfactants 68 may be utilized in stain removal applications alone and/or in combination with additional components (e.g., additives and/or linear or substantially linear alcohols) to generate alternative compositions. By way of example, referring to the method 60, at block 70, to form a detergent composition 74, one or more additives 72 may be provided to surfactants 68. As described herein, the additives 72 may include stabilizers (e.g., EDTA), alkali additives (e.g., NaOH), additional surfactants, or a combination thereof. In general, the disclosed surfactants 68 exhibit properties such as an average carbon number, branching index, and a defined distribution of the first branch position based on LBAs 32, which may improve stain removal performance when combined with additives 72 to produce detergent composition 74.

Accordingly, the detergent compositions 74 include about 0.5% to about 90% by weight of surfactant 68. The structure of surfactant 68 is represented by R—X, where R is the hydrocarbon moiety of the LBAs 32 (i.e. excluding —OH) and X is a hydrophilic moiety (e.g., surfactant precursor 62). For example, the detergent composition 74 may include surfactant 68, wherein the weight of the disclosed surfactant 68 based on the total weight of the detergent composition may range between about 2% to about 60%, about 3% to about 40%, about 5% to about 25%, or about 10% to about 25%.

Accordingly, X of R—X (e.g., surfactant precursor 62) may be selected from the group consisting of sulfates, sulfonates, amine oxides, polyoxyalkylene, polyhydroxy moieties, phosphate esters, polyphosphate esters, sulfosuccinates, polyalkoxylated carboxylates, glycerol esters, glycerol ester sulfates, polyglycerol ethers, polyglycerol ether sulfates, glycerol ethers, glycerol ether sulfates, sulfonated fatty acids, and mixtures thereof. X may be selected from the group consisting of sulfates, sulfonates, polyoxyalkylene, polyhydroxy moieties, amine oxide, glycerol ethers, glycerol ether sulfates, polyglycerol ethers, polyglycerol ether sulfates, and mixtures thereof. For example, X may be a sulfate or ethoxylate.

Alternatively, when X (e.g., surfactant precursor 62) is an anionic head group, the resulting anionic surfactant may exist in an acid form. Further, the acid form may be neutralized using a neutralization agent to form a surfactant salt. Example neutralization agents for neutralization of the acid form include metal counterion bases, such as hydroxides, e.g., NaOH, KOH, or. Additionally or alternatively, suitable neutralization agents may also include ammonia, amines, or alkanolamines. Non-limiting examples of alkanolamines include monoethanolamine, diethanolamine, triethanolamine, and other linear or branched alkanolamines known in the art; suitable alkanolamines include 2-amino-1-propanol, 1-aminopropanol, monoisopropanolamine, or 1-amino-3-propanol. It should be noted that neutralization using an amine may be carried out to any sufficient degree of completion. For example, the degree of completion may be 100%, 80%, 70%, 60%, 50%, and so on. As one non-limiting example, part of the anionic surfactant mix may be neutralized with sodium or potassium and part of the anionic surfactant mix may be neutralized with amines or alkanolamines.

In some instances, a portion of the carbon content of the surfactant may be derived from renewable sources. For example, about 0.1% to about 100% of the carbon content of the first surfactant may be derived from renewable sources such as used cooking oil, vegetable oil, palm kernel oil, corn/sugar cane ethanol etc. As used herein, a “renewable sources” refers to a feedstock that contains renewable carbon content, which may be assessed through ASTM D6866, which allows the determination of the renewable carbon content of materials using radiocarbon analysis by accelerator mass spectrometry, liquid scintillation counting, and isotope mass spectrometry.

The detergent compositions 74 may include one or more additives 72 (e.g., a second surfactant, a third surfactant) as selected from the group consisting of anionic surfactants, nonionic surfactants, cationic surfactants, zwitterionic surfactants, amphoteric surfactants, ampholytic surfactants, and mixtures thereof. The additional surfactant may be a detersive surfactant, which those of ordinary skill in the art will understand to encompass any surfactant or mixture of surfactants that provide cleaning, stain removing, or laundering benefit to soiled material.

### Surfactant Blending Compositions

In another embodiment, surfactants 68 may be combined with additional components to produce alternative compositions. By way of example, referring to the method 60, at block 76, to form a surfactant blend composition 80, one or more substantially linear alcohol derived surfactants 78 may be provided to surfactants 68. The substantially linear alcohol derived surfactants 78 may be a carbon length of C12, C13, C14, C15, or C16 and exhibit an average number ranging between about 12.5 to about 15.5. As referred to herein, “substantially linear” (e.g., very lightly branched) with respect to alcohols refers to alcohols having a branching index that is less than about 0.8, 0.5, or 0.3. It is presently recognized that blending surfactants 68 produced using the disclosed LBA 32 with a linear and/or substantially linear alcohol derived surfactants 78 (e.g., substantially linear alcohol derived sulfate, substantially linear alcohol derived alkoxylates, or substantially linear alcohol derived alkoxylate sulfates) having a different carbon number exhibit an improved cleaning performance through a synergistic effect.

As such, the surfactant blend composition 80 may have a surfactant ratio (e.g., weight (wt) % of the one or more surfactants 68 to the wt % of the one or more substantially linear derived surfactants 78 (e.g., substantially linear C₁₂-C₁₆ alcohols derived surfactants 78) that is greater than 0.5, about 1:1, about 2:1, about 3:1, about 4:1, or about 5:1. In other embodiments, the LBA 32 may be first blended with the substantially linear alcohol and subsequently undergo sulfation, alkoxylation, or a combination of alkoxylation and sulfation, thereby producing the surfactant blend compositions 80 (e.g., alcohol alkoxylates, alcohol derived sulfates, other surfactants that may be known in the art).

### EXAMPLES

### LBA Examples

With the foregoing in mind, Table 1 shows properties of the disclosed LBAs 32 and baseline composition alcohols. Referring to Table 1, Table 1 shows the disclosed branched C13 alcohol (e.g., Experimental Material 1 (Exp. Mat. 1 oxo alcohol or Exp. Mat. 1) corresponding to the disclosed LBAs 32. It should be noted that Exp. Mat. 1 oxo alcohol and Exp. Mat. 1 may be used interchangeably herein. Further, Table 1 shows comparative branched C13 alcohols (e.g., Reference Material 1 (Ref. Mat. 1 oxo alcohol or Ref. Mat. 1), Ref. Mat. 2 oxo alcohol or Ref. Mat. 2, Ref). It should be noted that Ref. Mat. 1 or Ref. Mat. 1 oxo alcohol may be used interchangeably.

| | Exp. | Ref. | Ref. |
|---|---|---|---|
| Alcohol characteristics | Mat. 1 | Mat. 1 | Mat. 2 |
| Kinematic viscosity mm²/s at 20° C. | 35.1 | 35.1 | 40.7 |
| Branching index | 1.53 | 1.83 | 2.1 |
| 1st Branch* distribution | | | |
| 2 | 12.17% | 30.59% | 27.70% |
| 3 | 28.11% | 27.12% | 13.90% |
| 3,4-disubstituted | 0 | 2.63% | 5.2% |
| 4 | 12.75% | 10.63% | 26.30% |
| 5&5+ | 46.97% | 29.03% | 26.90% |

Table 1 shows properties of the disclosed LBAs 32 (Exp. Mat. 1) and comparative branched C13 alcohols (Ref. Mat. 1 and Ref. Mat. 2).

The Exp. Mat. and Ref. Mat. 1 has a lower kinematic viscosity than Ref. Mat. 2. Also Exp. Mat. 1 has a lower BI than all the other reference branched C13 alcohols. For the 1^{st} branch distribution, Exp. Mat. 1 has a lot less position 2 branching and a lot more position 5 & 5+ compared with all the reference branched C13 alcohols.

Table 1 shows properties of the disclosed LBAs 32 and comparative branched C13 alcohols, wherein the properties include kinematic viscosity in mm²/s at 20° C., branching index, and distribution of the first branch position. For example, the kinematic viscosity (mm²/s) at 20° C. for the disclosed LBAs 32 may range between about 33 mm²/s and about 37 mm²/s, about 33 mm²/s, about 34 mm²/s, about 35 mm²/s, about 36 mm²/s, or about 37 mm²/s.

In particular, the BI of the LBAs 32 may range between about 1.3 to about 1.7. For 30 example, the BI of LBAs 32 may range between 1.4 and 1.6, about 1.3, about 1.4, about 1.5, about 1.6, or about 1.7. Further, the % distribution of LBAs 32 at exhibiting a first branch at position two 50 may range between 12% and 18%, 14% and 16%, about 10%, about 11%, about 12%, about 13%, about 14%, about 15%, about 16%, about 17%, about 18%, about 19%, or about 20%. The % distribution of the LBAs 32 at exhibiting a first branch at position three 52 may range between 22% and 38%, 24% and 36%, 26% and 34%, 28% and 32%, about 20%, about 22%, about 24%, about 26%, about 28%, about 30%, about 32%, about 34%, about 36%, about 38%, or about 40%. The % distribution of the LBAs 32 at exhibiting a first branch at position four 54 may range between 7% and 13%, 9% and 11%, about 5%, about 6%, about 7%, about 8%, about 9%, about 10%, about 11%, about 12%, about 13%, about 14%, or about 15%. Further, the % distribution of the LBAs 32 at exhibiting a first branch at position 5 & 5+ 56 may range between 37% and 53%, 39% and 51%, 41% and 49%, 43% and 47%, about 35%, about 37%, about 39%, about 41%, about 43%, about 45%, about 47%, about 49%, about 51%, about 53%, or about 55%.

LBAs 32 exhibit an include advantageous properties, or combinations of properties, such as branching index, viscosity, distribution of first branch position, or for use as a feedstock for producing surfactants.

### Surfactant Compositions, Reaction Conditions, and Examples

### Preparation of Alcohol Sulfates

The LBAs 32 are pre-cooled to less than 10° C. in a round bottom flask with an ice-salt water bath. Chlorosulfonic acid is added dropwise to the alcohol with dry N2 purge while maintaining the temperature between 5° C. to 15° C. The addition of chlorosulfonic acid was completed in about three hours. The salt water-ice bath was replaced with an ice water bath at 22-24° C., and the N2 purge continued for another 30 mins. The reaction product was then added slowly to a chilled 25% sodium methoxide solution in anhydrous methanol to convert the reaction product from an acid sulfate form to a sodium sulfate salt form. Additional anhydrous methanol is added when it is desirable to maintain neutralization product fluidity. The resulting mixture is cloudy, pale yellow in color, fluid, and mixing well. The product was mixed thoroughly for another two hours until a roughly 5% aqueous solution pH is stable between 9. 0 and 10.0. The reaction mixture is then poured into a plastic tray to evaporate the methanol in fume hood. The product was then put in a vacuum oven to further remove the solvents. The final dried product is off-white, soft solid.

### Preparation of Alcohol Ethoxylates

Dried LBAs 32 and catalyst mix are introduced to a reactor, and the reactor is heated to the desired reaction temperature. The reactor was pressurized with N2 for inertness. Further, the process includes starting a mass-controlled ethylene oxide (EO) feed to the reactor. After feeding the desired mass of EO, the process includes maintaining the reaction temperature for a specified time corresponding until most of the EO present in the reactor has reacted. Further still, the process includes depressurizing the reactor and start N2 purging to remove trace amounts of unreacted EO. Subsequently, the process includes neutralizing the catalyst and cooling to room temperature, and discharging the reactor.

Further embodiments of the present disclosure are directed to using the LBAs for stain removal and cleaning applications. It is presently recognized that the disclosed surfactants 68 (e.g., alcohol sulfates and alcohol ethoxylates) made from LBAs 32 demonstrate improved cleaning performance, as described in more detail below.

To test the cleaning performance using the disclosed LBAs 32, technical stain swatches of were purchased from TESTFABRICS. Detergency experiments were carried out using a 2 liter (L) jacked flask connected a circulation bath. A 15-minute wash cycle was performed with 1-liter detergent solution and 3 pieces of stain swatches. A 2-minute rinse was performed with 1-liter of deionized water following the washing. Washing and rinsing cycles were performed at 150 revolutions per minute (rpm) agitation speed. The bath temperature was kept constant at 20° C. during the washing and rinsing cycles. Finally, the washed fabric blends were hung in a fume hood overnight to dry. Detergency was determined by measuring the L, a and b value using a Hunter Lab-Colorflex-Ez spectrophotometer based on a modified ASTM D3050-07method.

$$SRI = \sqrt{{( {L}_{w a s h e d} - {L}_{u n w a s h e d} )}^{2} + {( {a}_{w a s h e d} - {a}_{u n w a s h e d} )}^{2} + {( {b}_{w a s h e d} - {b}_{u n w a s h e d} )}^{2}}$$

$$Δ SRI = SRIsample - SRIcontrol$$

Alcohol Ethoxylates

| | Δ SRI | |
|---|---|---|
| | Alcohol ethoxylate | Disclosed C₁₃ alcohol |
| | with 7EO of Ref. | ethoxylate with 7EO |
| Stains | Mat. 1 (control) | of Exp. Mat. 1 |
| Grass | 0.00 | 0.3 |
| Make-up | 0.00 | 3.24 |
| Mineral Oil with | 0.00 | 1.32 |
| carbon black | | |
| Egg yolk | 0.00 | 2.96 |
| Sebum with pigment | 0.00 | 1.99 |

Table 2 shows ΔSRI of the alcohol ethoxylates at 400 ppm in deionized (DI) water.

Table 2 shows the stain removal index (SRI) of alcohol ethoxylates of 7EO produced using the disclosed LBAs 32 vs comparative branched C13 alcohol (Ref. Mat. 1) Accordingly, alcohol ethoxylates produced using the disclosed LBAs 32 exhibit improved stain (e.g., grass, makeup) removal performance relative to the comparative compositions.

### Detergent Compositions

| | Composition A, | Composition B, |
|---|---|---|
| Formulation ingredients | ppm (control) | ppm |
| Disclosed C13 alcohol derived | 0 | 360 |
| sulfate of Exp. Mat. 1 | | |
| Alcohol sulfate of Ref. Mat. 2 | 360 | 0 |
| LAS (linear alkyl benzene | 140 | 140 |
| sulfonate) | | |
| C12-16 alkyl ethoxylate (EO7) | 140 | 140 |
| AMMONYX LO | 20 | 20 |
| SOKALAN HP 20 | 30 | 30 |

Table 3 shows an example composition used for the alcohol derived sulfate laundry cleaning performance test.

By way of example, Table 3 shows example detergent compositions prepared using the disclosed LBAs 32. Table 3 shows a first type of conditions (e.g., Composition B) corresponding to a composition including surfactant 68 (e.g., alcohol derived sulfate) produced using the disclosed LBAs 32. Further, Table 3 shows a second type of conditions that are the comparatives (e.g., Composition A) produced using comparative C₁₃ oxo alcohols (Ref. Mat. 2 oxo alcohol). The detergent compositions were tested at 120 ppm water hardness and 20° C., as demonstrated in Table 3.

ΔSRI

| | Composition A | Composition B |
|---|---|---|
| Mineral Oil with carbon black | 0 | 1.3 |
| Sebum with pigment | 0 | 1.4 |
| Make up | 0 | 2.1 |
| Tea | 0 | 1.9 |

Table 4 shows the ΔSRI of Composition A and Composition B.

Laundry cleaning performance test of Composition A and Composition B was evaluated on various stains in Table 4. Accordingly, Composition B, which includes alcohol derived sulfates produced using the disclosed LBAs 32, exhibits improved stain removal performance of various stains (e.g., mineral oil with carbon black, sebum with pigment, makeup, tea) relative to the comparative compositions (e.g., Composition B). Therefore, the detergent compositions produced using the disclosed LBAs 32 described herein provide advantageous stain removal properties relative to comparative compositions.

| | Composition | Composition |
|---|---|---|
| | C | D (control) |
| Soap (Coconut fatty acid 745) | 0.60% | 0.60% |
| Nacconol 90G | 2.42% | 2.42% |
| Disclosed C13 alcohol derived | 2.20% | |
| ethoxylate with 7EO of Exp. Mat. 1 | | |
| Alcohol ethoxylate with 7EO of | | 2.20% |
| Ref. Mat. 1 | | |
| EDTA, 100% active | 0.15% | 0.15% |
| Citric acid, 100% active | 0.30% | 0.30% |
| NaOH, 10% active | 3.21% | 3.21% |
| Distilled H2O | 91.12% | 91.12% |
| Total | 100.00% | 100.00% |

Table 5 shows example compositions used for the alcohol derived ethoxylates laundry cleaning performance test.

By way of example, Table 5 shows example detergent compositions used for the alcohol derived ethoxylates laundry cleaning performance test. In general, Table 5 shows an example detergent composition used for the alcohol derived ethoxylate laundry cleaning performance test. Table 6 shows a first type of conditions (e.g., Composition C) corresponding to a composition including surfactant 68 (e.g., alcohol derived ethoxylate) produced using the disclosed LBAs 32. Further, Table 6 shows a second type of condition that is the comparative composition (e.g., Composition D) produced using the comparative C₁₃ oxo alcohols (Ref. Mat. 1 oxo alcohol). The detergent compositions were tested at 120 ppm water hardness and 20° C. at a total surfactant concentration (soap+Nacconol 90G+alcohol derived ethoxylates) of 500 ppm.

| | ΔSRI | |
|---|---|---|
| | | Disclosed C₁₃ alcohol |
| | Alcohol ethoxylate with | derived ethoxylate with |
| | 7EO of Ref. Mat. 1 (control) | 7EO of Exp. Mat. 1 |
| Make up | 0 | 9.1 |
| Tea | 0 | 2.4 |

Table 6 shows ΔSRI of the compositions of Table 5.

Laundry cleaning performance test of Composition C and Composition D was evaluated on various stains, as shown in Table 6. Accordingly, Composition C, which includes alcohol derived ethoxylates produced using the disclosed LBAs 32, exhibits improved stain removal performance of various stains (e.g., makeup, tea) relative to the comparative compositions (e.g., Composition D). Therefore, the detergent compositions produced using the disclosed C₁₃ oxo alcohol 32 (e.g., disclosed LBA 32) described herein provide advantageous stain removal properties relative to baseline compositions.

By way of example, Table 7 illustrates stain removal performance of alcohol derived sulfates produced using the disclosed C₁₃ oxo alcohol 32 compared to an example of a surfactant blend composition 80, wherein the surfactant blend composition 80 includes a combination of alcohol derived sulfates produced using the disclosed LBAs 32 and C₁₅ alcohol derived sulfates (i.e., a substantially linear C₁₅ alcohol derived sulfate 78). The surfactant blend composition 80 includes a ratio of the disclosed C₁₃ alcohol derived sulfate and C₁₅ alcohol derived sulfate at 4:1. The C₁₅ alcohol has a branching index of 0.25. The pure C₁₅ alcohol derived sulfate was not tested for detergency as it was not soluble at 400 ppm concentration.

ΔSRI

| | | Disclosed C₁₃ alcohol |
|---|---|---|
| | Disclosed C₁₃ | derived sulfate of |
| | alcohol derived | Exp. Mat. 1/C₁₅ alcohol |
| | sulfate of Exp. | derived sulfate |
| | Mat. 1 | blend at 4:1 |
| Grass | 0 | 3.6 |
| Make up | 0 | 2.4 |
| Coffee | 0 | 3.2 |
| Mineral Oil with | 0 | 1.9 |
| carbon black | | |
| Egg yolk | 0 | 2.6 |
| Sebum with pigment | 0 | 0.2 |

Table 7 shows the ΔSRI of the disclosed C₁₃ alcohol derived sulfate compared to a sulfate blend (e.g., disclosed C₁₃ alcohol derived sulfate and a substantially linear C₁₅ alcohol derived sulfate) at 400 ppm in DI water.

Accordingly, Table 7 shows that the surfactant blend composition 80 (e.g., disclosed C₁₃ alcohol derived sulfate and a substantially linear C₁₅ alcohol derived sulfate) exhibits surprising improved stain removal performance of various stains (e.g., mineral oil with carbon black, sebum, makeup, tea) relative to the disclosed C₁₃ alcohol derived sulfate alone. Therefore, the sulfate blends produced using the disclosed LBAs 32 and C₁₅ alcohol derived sulfate described herein provide advantageous stain removal properties relative to baseline compositions.

With the foregoing in mind, Table 8 shows stain removal performance of alcohol derived sulfates produced using the disclosed LBAs 32 (e.g., Composition E) compared to an example of a surfactant blend composition 80 blend (e.g., Composition F), wherein the surfactant blend composition 80 includes a sulfate blend with a combination of disclosed C₁₃ alcohol derived sulfates and linear C₁₄ alcohol derived sulfates at a ratio of 3:1. Table 8 also includes stain removal data of the linear C₁₄ alcohol derived sulfate (e.g., Composition G) alone.

| | ΔSRI | | |
|---|---|---|---|
| | Composition E | Composition F (Disclosed C₁₃ | Composition G |
| | (Disclosed C₁₃ alcohol | alcohol derived sulfate of Exp. | (linear C₁₄ |
| | derived sulfate of | Mat. 1/linear C₁₄ alcohol derived | alcohol derived |
| | Exp. Mat. 1) | sulfate blend at 3:1) | sulfate) |
| Mineral Oil with | 0 | 0.8 | 0.1 |
| carbon black | | | |
| Egg yolk | 0 | 1.0 | Not tested |
| Grass | 0 | 2.7 | −0.1 |

Table 8 shows ΔSRI of compositions of the disclosed C₁₃ alcohol derived sulfate compared to a sulfate blend (e.g., disclosed C₁₃ alcohol derived sulfate and linear C₁₄ alcohol derived sulfate) and a linear C₁₄ alcohol derived sulfate based on formulas as described in Table 6.

Table 8 shows that Composition F exhibits surprising improved stain removal performance of various stains (e.g., mineral oil with carbon black, egg yolk and grass) relative to the disclosed C₁₃ alcohol derived sulfate on its own and the linear C₁₄ alcohol derived sulfate on its own. In particular, it is noted that the disclosed C₁₃ alcohol derived sulfate (e.g., branched C₁₃ alcohol derived sulfate) produced using the LBAs 32, and in combination with one or more linear and/or substantially linear C₁₂-C₁₆ alcohol derived surfactants demonstrates improved cleaning performance as compared to the branched C₁₃ alcohol derived sulfate alone (e.g., not in combination with other C₁₃ or greater alcohols) or the linear and/or substantially linear C₁₂-C₁₆ alcohol derived surfactants alone (e.g., not in combination with other C₁₃ or greater alcohol derived surfactants). Therefore, the surfactant blend compositions 80 described herein provide advantageous stain removal properties relative to individual surfactants.

Accordingly, aspects of the present disclosure are directed to techniques for producing LBAs 32 using butene and an optional propylene and subsequently utilizing the LBAs 32 and reacting it with surfactant precursors to produce surfactants. In this way, a surfactant precursor feedstock in the presence of the LBA may produce a surfactant stream having certain physical properties, such as limited branching and first branch distribution that are useful for surfactant-based applications such as good cleaning performance and stain removal properties in cold water. Further, the alcohol could be derivatized to esters, acrylates for coatings, adhesives, lubricants applications. The surfactants 68 described herein may be used in a variety of consumer and industrial products, including, but not limited to, detergents, emulsifiers, cosmetics, pharmaceuticals, dispersants, home and personal care areas like hand and auto dishwashing, hard surface cleaning, body washing, shampoo and industrial applications such as textile, agriculture, emulsion polymerization, metal working fluids. As described above, the disclosed alcohol derived ethoxylate surfactant and alcohol derived sulfate surfactant may exhibit a good balance of good cleaning performance and stain removal properties in cold water.

### Characterization of the Lightly Branched Alcohols

Additional aspects of this disclosure related to techniques for determining branching properties of long chain alcohols, and generating outputs (e.g., control signals, alerts, visualizations) based on the determined branching properties. Long chain alcohols (e.g., higher alcohols greater than or equal to 7 carbons) are utilized in numerous of industrial applications. The application of the long chain alcohol is a function of properties (e.g., chemical structure) of the long chain alcohol. For example, the branching of alcohols has been shown to affect certain physicochemical parameters of formulations derived from or otherwise composed of the alcohols. These physicochemical parameters include, among others, curvature values, which is an indication of hydrophobicity, and is often utilized for formulation applications. Although properties of small alcohols have been characterized, the properties of long chain alcohols are more difficult to characterize due to, for example, the complexity of interactions between the long chain alcohols. For example, alcohols are known for their ability to hydrogen bond, which is often non-linear and complex. As the alkyl chain of an alcohol increases in length, behaviors associated with small alkyl alcohols may not be applicable due to the non-negligible dispersive interactions of the alkyl chains. Instead, higher alcohols may be more affected by parameters such as temperature, pressure, steric hindrance of groups adjacent to the hydroxyl group, the balance of polar and nonpolar interactions, etc. Conventional techniques such as gas chromatography (GC) and nuclear magnetic resonance (NMR) spectroscopy may be leveraged to elucidate properties (e.g., branching) of long chain alcohols. However, despite their accuracy and reliability, GC and NMR are cost prohibitive, are associated with complex sample preparation, and often involve time-intensive procedures, thereby necessitating alternative analytical techniques that can be implemented on-, in- or at-line in a process or quality control environment.

Vibrational spectroscopy (e.g., Raman, Fourier transform infrared (FTIR), near infrared spectroscopy (NIR)) may be used to characterize and quantify the structure and behavior of small alcohols. For example, vibrational spectroscopy is utilized to identify the region corresponding to the hydroxyl band. However, there is currently a need for improved FTIR techniques for determining branching properties of long chain alcohols. With the foregoing in mind, Fourier transform infrared (FTIR)-attenuated total reflectance (ATR) (FTIR-ATR) spectroscopy is a promising technique that may be utilized to characterize certain long chain alcohols, such as lightly branched alcohols. The disclosed techniques may include characterization of the lightly branched alcohols (e.g., LBAs 32, higher alcohols, long chain alcohols) using FTIR-ATR spectroscopy to determine branching index (e.g., average number of branches, percentage of molecules with their first branch at the second (2nd) position). In particular, the disclosed techniques may leverage the fingerprint region of the IR-spectrum (e.g., wavenumbers ranging 1600 and 600 cm⁻¹) to elucidate behaviors of long chain alcohols to determine their branching. In one embodiment, the techniques include determining the average number of branching using spectral peaks associated with CH₂ and CH₃ vibrations and determining branching at the 2^{nd} position using a spectral peak associated with C—C—O vibrations, respectively. In another embodiment, a linear regression model (e.g., correlation model, linear calibration model) is generated by correlating FTIR-ATR spectra with NMR spectra (e.g., ¹H NMR, ¹³C NMR) to determine branching and branching at the 2nd position of the lightly branched alcohols (e.g., LBA 32). It should be noted that present techniques is also suitable for characterizing other molecules (e.g., substantially linear molecules, highly branched molecules) as well. For example, the FTIR characterization methods described herein may be suitable for a branching index ranging from about 0.6 to about 3.1 and a broader carbon number range. In this way, the generated linear calibration model may be subsequently utilized to determine branching and branching at the 2nd position of long chain alcohols. The disclosed techniques in combination with the generated linear calibration model provides several advantages such as enabling a cost-effective analysis, eliminating sample preparation, and can be implemented on-, in- or at-line in a process/quality control environment.

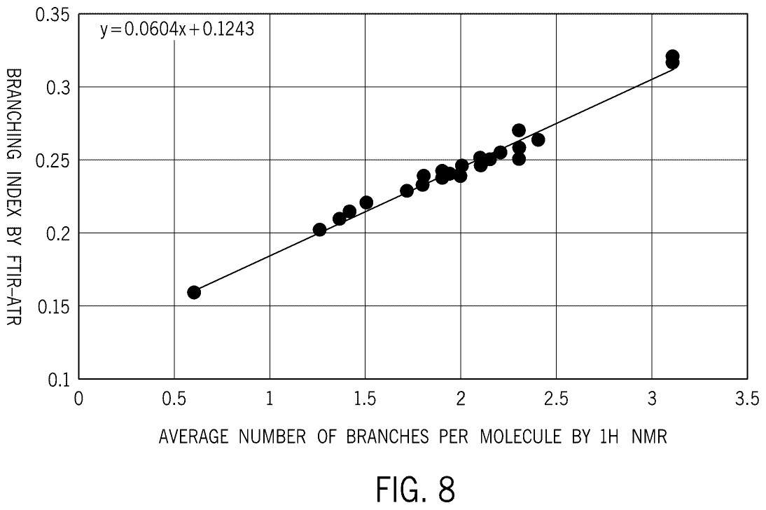

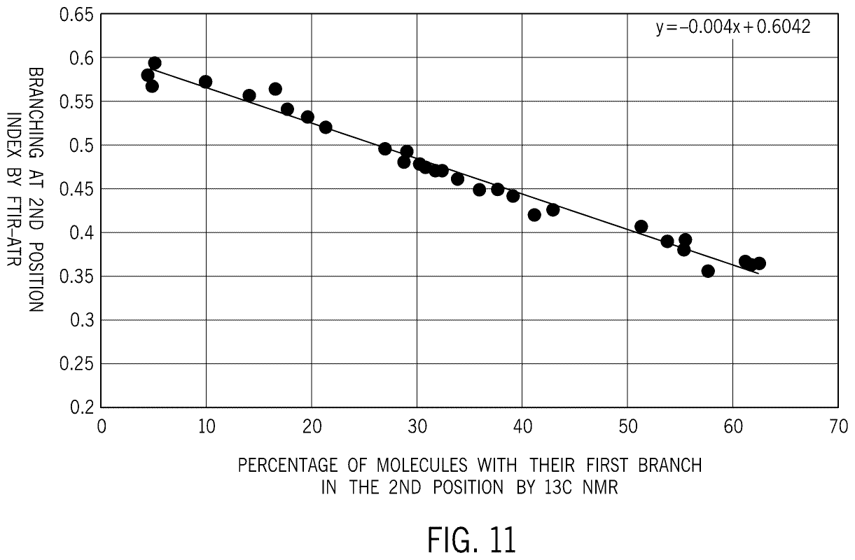

To illustrate the disclosed techniques, branching properties (e.g., branching index, branching at 2nd position) of a set of 37 branched alcohols (including lightly branched alcohol LBA 32) that were measured via NMR spectroscopy (e.g., ¹H NMR, ¹³C NMR) are discussed herein. Table 9 shows examples of branched alcohol samples (e.g., Sample 1, Sample 2, Sample 3, Sample 4) and their respective branching index values and branching at 2^{nd} position, as determined by ¹H NMR and ¹³C NMR, respectively. In general, the four branched alcohols exhibit a branching index (average number of branches per molecule) that ranges from 0.6 to 3.1, as determined by ¹H NMR. The branching at 2^{nd} position of the four branched alcohols (e.g., percentage of molecules with their first branch in the 2^{nd} position) ranges from 5.1% to 62.3%, as determined by ¹³C NMR. For example, Table 9 shows that Sample 4 is substantially more linear relative to the other comparative samples (e.g., Sample 1, Sample 2, Sample 3). That is, Sample 4 has the least average number of branches per molecule, whereas Sample 1 has the greatest average number of branches per molecule. Additionally, Table 9 shows that Sample 4 has the least percentage of molecules with their first branch in the 2^{nd} position, while Sample 2 has the greatest percentage of molecules with their first branch in the 2^{nd} position. It should be noted that the samples in Table 9 are meant to be exemplary. Accordingly, correlation models (as described in FIGS. 8 and 11 below below) may employ several samples to generate the correlation models.

| | | Branching at 2^{nd} |
|---|---|---|
| | Branching index | position (percentage |
| | (average number of | of molecules with |
| | branches per | their first branch |
| Sample Code | molecule) | in the 2^{nd} position) |
| Sample 1 | 3.1 | 9.9% |
| Sample 2 | 2.3 | 57.6% |
| Sample 3 | 2.2 | 19.6% |
| Sample 4 | 0.6 | 5.1% |

Table 9 shows examples of branched alcohol samples and their respective branching index values and branching at 2^{nd} position.

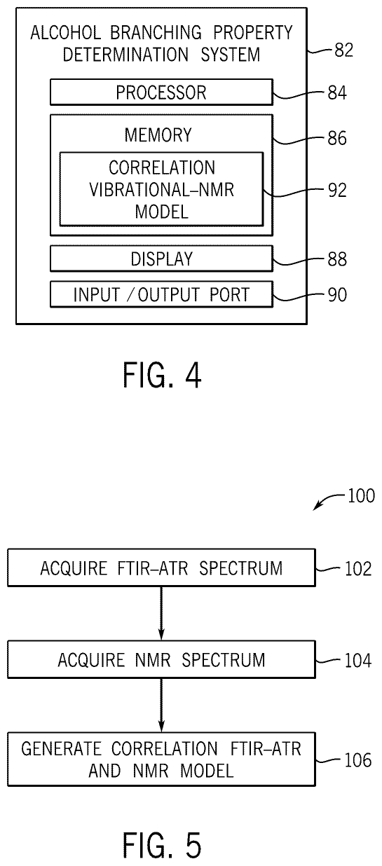

The disclosed techniques for determining branching properties of long chain alcohols may be performed by an alcohol branching property determination (ABPD) system 82 shown in FIG. 4. The ABPD system 82 may include a processor 84, memory/storage 86, a display 88, input/output (I/O) port 90, and the like, in accordance with embodiments described herein.

The processor 84 may be any type (e.g., a physical processor and a virtual processes) of computer processor or microprocessor capable of executing computer-executable code. The processes described in this specification may be performed by one or more programmable processors executing one or more computer programs to perform functions by operating on input data (e.g., measurements from a chemical production system) and generating output. The processes and logic flows may also be performed by special purpose logic circuitry, e.g., an FPGA (field programmable gate array) or an ASIC (application specific integrated circuit). Processors suitable for the execution of a computer program include, by way of example, both general and special purpose microprocessors, and/or processor(s) of any appropriate kind of digital computer.

The memory and the storage 86 may be any suitable articles of manufacture that store processor-executable code, data, or the like. These articles of manufacture may include non-transitory computer-readable media (e.g., any suitable form of memory or storage) that may store the processor-executable code used by the processor 84 to perform the presently disclosed techniques. It should be noted that non-transitory merely indicates that the media is tangible and not a signal. As illustrated, the memory 86 and/or the storage may store the correlation vibrational-NMR model 92 (e.g., IR). The vibrational-NMR correlation model 92 (e.g., IR) may store correlations between infrared spectral regions and NMR data of certain long chain alcohols that may be used to determine branching properties of the long chain alcohols, such as lightly branch alcohols.

The display 88 may depict visualizations associated with software or executable code being processed by the processor 84, such as a graph visualization indicate IR speaks that correspond to a long chain alcohol. The display 88 may be any suitable type of display, such as a liquid crystal display (LCD), plasma display, or an organic light emitting diode (OLED) display, a projected display, and the like. Additionally, in one embodiment, the display 88 may be provided in conjunction with a touch-sensitive mechanism or touch display (e.g., a touch screen) that may function as part of a control interface for the ABPD system 82 and be capable of receiving inputs from a user (e.g., an operator working with a chemical production system, such as a plant). The I/O ports 90 may be interfaces that may couple to other peripheral components such as input devices (e.g., keyboard, mouse), and the like. For example, to provide for interaction with a user, implementations may be realized on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer.

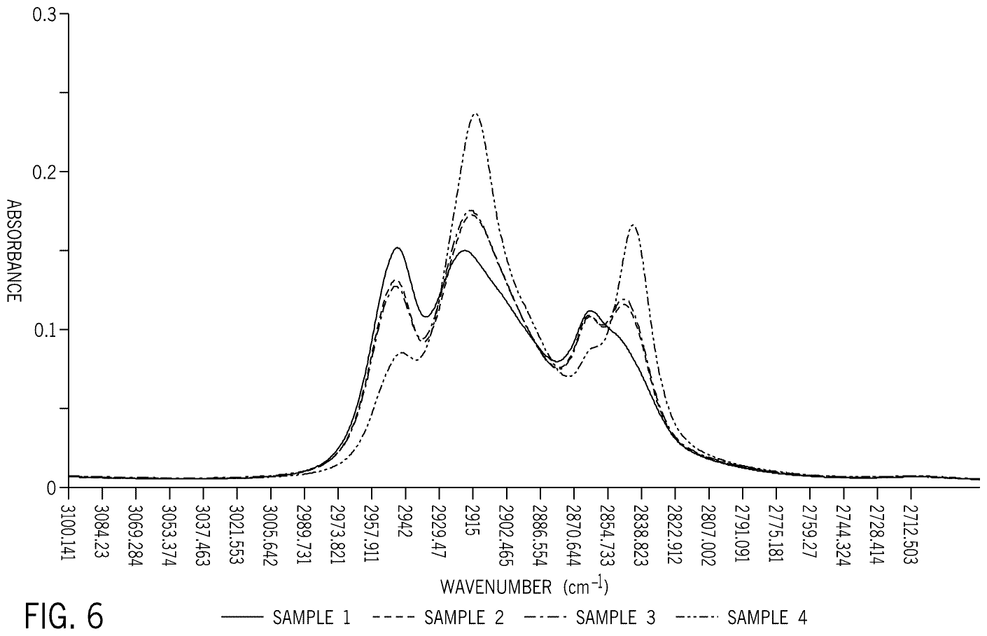

FIG. 5 flow diagram of a method 100 for generating a correlation linear regression model including Fourier transform infrared-attenuated total reflectance (FTIR-ATR) and nuclear magnetic resonance (NMR) data. It should be noted that the example method 100 shown in FIG. 5 is not limiting, and the method 100 may include additional or fewer blocks than those illustrated. Further, the method 100 may include block that are performed in an alternative order to that illustrated. That is, certain blocks may be performed before, after, or concurrently to/with another respective step. Although the blocks are described as being performed by the processor 84, it should be noted that the any number of processors may perform the blocks of the method 100.

Referring to FIG. 5, at block 102, the processor 84 acquires an FTIR-ATR spectrum of a sample (e.g., a solution) including one or more long chain alcohols (e.g., lightly branched alcohol, C13 alcohols). For example, the processor 84 may provide a control signal that causes an FTIR spectrometer to collect the IR spectrum.

The FTIR-ATR spectrum may include one or more spectral regions that correspond to vibrations that occur in alkyl chains and alcohol vibrations (e.g., C—H bends, C—C—O vibrations, and the like), such as between about 600 cm⁻¹ and about 1600 cm⁻¹ wavenumbers. In some embodiments, the processor 84 may determine or select a spectral region for the FTIR-ATR spectrum based on input provided by a user or sensors measuring the presence of chemical components or other properties (e.g., pressure, temperature) within a chemical production system. In general, the processor 84 may select one or more spectral ranges that correspond to a particular vibration of interest. In some embodiments, the processor 84 may utilize a reference table (e.g., stored in the memory 86) that stores information relating certain peaks to certain alcohols, and so on.

As one non-limiting example, the processor 84 may select a spectral region ranging from about 1330 cm⁻¹ to about 1480 cm⁻¹ and identify structural peaks within the spectral region that are associated with the alcohols to determine a branching index. It may be advantageous to utilize the spectral region from 1330 cm⁻¹ to about 1480 cm⁻¹ since it may include peaks that represent CH₂ deformation (scissors) and CH₃ bending vibrations, which correspond to wavenumbers at about 1465 cm⁻¹ and about 1378 cm⁻¹, respectively. After peak identification, the processor may analyze the FTIR-ATR to determine the branching index by using a respective formula (e.g., AR_{Branching} formula). In some embodiments, the processor 84 may output a control signal to one or more flow control devices (e.g., valves, pumps, and the like) that causes a sample to be diverted from a reaction vessel, flow conduit, or otherwise, to a spectrometer capable of acquiring an FTIR measurement of the sample.

As another non-limiting example, the processor 84 may select a spectral region ranging from about 936 cm⁻¹ to 1140 cm⁻¹ and identify structural peaks associated with the alcohols to determine a percentage of molecules with their first branch at the 2nd position. It may be advantageous to utilize the spectral region from 936 cm⁻¹ to 1140 cm⁻¹ since it may include peaks that represent C—C—O vibrations, which correspond to wavenumbers ranging from about 1000 cm⁻¹ to about 1100 cm⁻¹. That is, the number of branches (i.e., degree of branching) may shift the wavenumber corresponding to C—C—O vibrations. The processor 84 may subsequently process the FTIR-ATR spectrum to facilitate identification and quantification of the long chain alcohols. For example, the processor 84 may perform a Fourier deconvolution for signal processing (e.g., Fourier self-deconvolution (FSD)) to resolve various peaks within the acquired FTIR-ATR spectra. After peak identification, the processor 84 may analyze the FTIR-ATR and determine the percentage of molecules with their first branch in the 2^{nd} position by using a respective formula (e.g., IR_{Branching at 2nd position} formula).

At block 104, the processor 84 obtains, measures, or otherwise acquires an NMR spectrum. For example, the processor 84 may provide a control signal that causes to collect a sample to obtain the NMR spectrum. In certain embodiments, the processor 84 may acquire an ¹H NMR spectrum and determine an average number of branches per molecule (e.g., branching index) using the ¹H NMR spectrum. In other embodiments, the processor may utilize a ¹³C NMR spectrum to determine a percentage of molecules with their first branch in the 2^{nd} position.

At block 106, the processor generates a correlation FTIR-ATR and NMR model (e.g., the correlation vibrational-NMR model 92) using the FTIR-ATR data and NMR data (e.g., ¹H NMR data, ¹³C NMR data). The FTIR-ATR and NMR model is a correlation linear regression model. The processor 84 may generate the linear regression model for branching index by plotting the data generated by the AR_{Branching} formula against the 1H NMR branching index data. The processor 84 may fit the correlated data to a linear regression model, thereby generating the correlation linear regression model. In this way, the processor 84 may utilize the generated linear regression model to estimate and quantify branching index for other alcohols using FTIR-ATR spectroscopy data. For example, the processor 84 may obtain FTIR-ATR data of different alcohols at a spectral region ranging from about 1330 cm⁻¹ to about 1480 cm⁻¹. The acquired spectral data may be inputted into the generated linear regression model (e.g., equation) to quantify a branching index for the different alcohols.

The processor 84 may generate the linear regression model for percentage of molecules with their first branch at the 2^{nd} position by plotting the data generated by the IR_{Branching at 2nd position} formula against the ¹³C NMR branching at 2^{nd} position data. In this way, the generated linear regression model for branching at 2^{nd} position may be subsequently utilized (e.g., by the processor 84) to estimate and quantify branching at 2^{nd} position for other alcohols using FTIR-ATR spectroscopy. For example, the processor 84 may acquire FTIR-ATR data of other alcohols at a spectral region ranging from about 936 cm⁻¹ to 1140 cm⁻¹. The processor 84 may provide the acquired spectral data as an input into the generated linear regression model (e.g., equation) and obtain an output indicating a quantification of a percentage of molecules with their first branch at 2^{nd} position for the other alcohols.

The linear regression model may be utilized by the processor 84 to generate outputs, such as a visualization indicating an amount of branching in a sample. In some embodiments, the processor 84 may generate a control signal that controls operation of a chemical production system. For example, the processor 84 may adjust a valve or reaction/process conditions based on a determination that the sample includes a branching index within a particular range, which indicate that solution (where the sample originated from) has a desired reaction product, or that the solution should be discarded.

Accordingly, the disclosed techniques may provide a fast and reliable method to unravel complex molecular structures of the long chain alcohols such as branching pattern of higher alcohols (e.g., branched alcohols, and, in particular, lightly branched alcohols) by leveraging the about 600 cm⁻¹ and about 1600 cm⁻¹ region. It should be noted other types of alcohols (e.g., different lengths, different degrees of branching) and/or other vibrational techniques (e.g., NIR, Raman spectroscopy) may also be utilized in generally similar manner. By way of example, Table 10 demonstrates band assignments of functional groups

present in branched alcohols, as determined via FTIR-ATR spectroscopy. For example, the branched alcohols may exhibit a broad OH stretching between about 3000 cm⁻¹ and about 3600 cm⁻¹ and CH stretching modes between about 2700-1 to about 3100 cm⁻¹. Table 10 further shows wavenumbers associated with the fingerprint region (e.g., less than 1600 cm⁻¹), which includes structural information associated with the branched alcohols. The fingerprint region is dominated by CH₂, CH₃ deformation, C—C, C—O, and C—C—O stretching modes. For example, the R atom in all alcohols (e.g., ROH) may exhibit two bands with maxima at about 1465 cm⁻¹ and about 1378 cm⁻¹, which correspond to CH₂ deformation (scissors) and CH₃ bending vibrations, respectively. Between about 1000 cm⁻¹ and about 1100 cm⁻¹, C—C—O peaks are present. It should be noted that the exact position of the C—C—O peaks may rely on the extent of branching at the 2^{nd} position (i.e., branching at R in R—C—OH, first branch at position two). Concomitantly, the linearity of an alcohol may affect spectra such that linear segment spectral bands may appear between about 720 cm⁻¹ and about 740 cm⁻¹ depending on the length of the alcohol. Given the amount of information present within the fingerprint region, it is presently recognized that the fingerprint region of the branched alcohols may be leveraged (e.g., by the processor 84) to determine branching properties.

| Wavenumber | | |
|---|---|---|
| (cm⁻¹) | Functional Group | Type of Vibration |
| 3340 | OH | Intermolecular hydrogen- |
| | | bonded O—H Stretch |
| 2954 | CH₃ | asymmetric stretch |
| 2923 | CH₂ | asymmetric stretch |
| 2871 | CH₃ | symmetric stretch |
| 2854 | CH₂ | symmetric stretch |
| 1465 | CH₂ | CH₂ deformation (scissors) |
| 1378 | CH₃ | CH₃ bending |
| ~1050 | R—CH₂—CH₂—OH | Out-of-phase C—C—O stretch |
| ~1035 | (R₁,R₂)CH—CH₂—OH | |
| ~1020 | (R₁,R₂,R₃)C—CH₂—OH | |
| 720-740 | (CH₂)ₙ | Rocking/Bending in plane |
| 700-600 | OH | Deformation out of Plane |

Table 10 shows band assignments of functional groups present in the branched alcohols, as determined via FTIR-ATR spectroscopy.

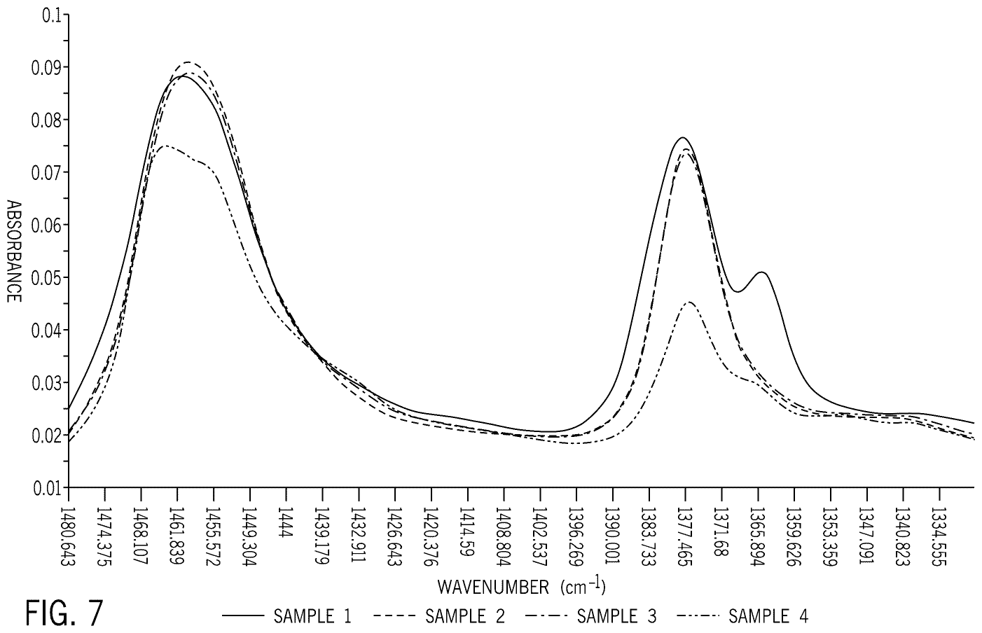

FIG. 6 is an example FTIR-ATR spectrum of branched alcohol samples in the 2700 cm⁻¹ to 3100 cm⁻¹ region. In some embodiments, FTIR spectra of the branched alcohols may be normalized with respect to the OH stretching band to facilitate identification of the CH band, which corresponds to the number of alkyl chains (e.g., branches). The y-axis is representative of absorbance (in reflection mode), and the x-axis is representative of the wavenumbers in cm⁻¹. FIG. 6 demonstrates spectral differences between the substantially the least branched sample (e.g., Sample 4) and the more highly branched alcohols (e.g., Sample 1, Sample 2, Sample 3) due to their respective chemical structures. In general, an increasing CH band (e.g., CH₃) may correspond to incremental increase in alkyl chain with due to the presence of branches. That is, the samples with relatively lower branching (e.g., Sample 4) exhibit a higher number of CH₂ groups relative to the other samples (e.g., Sample 1, Sample 2, Sample 3), while the samples with higher branching (e.g., Sample 1, Sample 2, Sample 3) exhibit a higher number of CH₃ groups. These results demonstrate that FTIR-ATR can distinguish lightly branched alcohol based on spectral peaks that correspond to a degree of branching.

### Branching Index

As shown in Table 10, the branched alcohols exhibit two bands with maxima at about 1465 cm⁻¹ and about 1378 cm⁻¹, which correspond to CH₂ deformation (scissors) and CH₃ bending vibrations, respectively. With the preceding in mind, FIG. 7 is an example of FTIR-ATR spectrum of branched alcohol samples in the 1330 cm⁻¹ to 1480 cm⁻¹ region. The y-axis is representative of absorbance (in reflection mode), and the x-axis is representative of the wavenumbers in cm⁻¹. In particular, FIG. 7 shows variations in the CH₂ deformation (scissors) and CH₃ bending vibrations between the four branched alcohol samples within the 1465 cm⁻¹ and about 1378 cm⁻¹, which is associated with the degree of branching for a respective sample. In general, the least branched sample, Sample 4, exhibits a weaker signal relative to the more highly branched samples (e.g., Sample 1, Sample 2, Sample 3). Due to the observed spectral differences, this spectral region may be used to determine the average molecular branching (i.e., branching index). In particular, the CH₃ group vibrations are associated with average molecular branching, and thus, the branching index (AR_{Branching}) was calculated using the formula (e.g., AR_{Branching} formula):

$$A {R}_{B r a n c h i n g} = \frac{{Area}_{CH 3}}{{Area}_{CH 2} + {Area}_{CH 3}} = \frac{{A}_{1378}}{{A}_{1465} + {A}_{1378}}$$

wherein Area_{CH3} is the area under a peak at about 1378 cm⁻¹ and Area_{CH2} is the area under the peak at about 1465 cm⁻¹, respectively. To validate the results of the branching index as determined by the FTIR-ATR spectra and the preceding formula, a linear calibration model was generated by correlating FTIR-ATR data and branching index (average number of branches per molecule) acquired via 1H NMR spectroscopy, which can be seen in FIG. 8.

FIG. 8 is a graph of a correlation model including FTIR-ATR and ¹H NMR data.

The correlation model includes a linear calibration model (e.g., linear regression model) that was generated using the AR_{Branching} values as determined from FIG. 7 and the preceding formula and branching index data (average number of branches per molecule) acquired via ¹H NMR (part of data presented on Table 9) of the branched alcohols. The y-axis represents branching index as measured by FTIR-ATR, while the x-axis represents average number of branches per molecule as determined by ¹H NMR. The linear calibration model demonstrates that there is a good correlation between FTIR-ATR and ¹H NMR spectroscopy data, which is further demonstrated in Table 11. These results demonstrate that the correlation model (e.g., the linear calibration model) may be utilized to facilitate estimation of the branching index of lightly branched alcohols using FTIR-ATR spectroscopy. That is, FTIR-ATR data may be acquired of other alcohols at a spectral region ranging from about 1330 cm⁻¹ to 1480 cm⁻¹. The acquired spectral data may be inputted into the generated linear regression model to quantify a branching index for the other alcohols. In this way, the present techniques facilitate a fast and reliable way to determine branching of lightly branched alcohols.

| | Branching |
|---|---|
| | Index Model |
| R² | 0.97 |
| RMSEC (Average number of branches per molecule) | 0.076 |
| RMSEV (Average number of branches per molecule) | 0.115 |

Table 11 shows estimated values of the linear calibration model as shown in FIG. 8. (R²: coefficient of determination, RMSE: root mean square error (C: calibration, V: validation))

The developed modeling approach correlating FTIR and ¹H NMR data using structural indexes (or chemometrics) is valid for spectral data of long chain alcohols (e.g., lightly branched C13 alcohols) analyzed by infrared spectroscopy in reflection (or in transmission measurement mode) having their branching index (e.g., average number of branches per molecule) that ranges from 0.6 to 3.1, as identified by ¹H NMR. While the methodologies disclosed herein utilize lightly branched C13 alcohols, a similar methodology may be employed to produce a linear calibration model that is specific for long chain alcohols that exhibit greater or lower average branching than the samples described herein or for different types of alcohols (e.g., alcohols greater than or lower than C13), or using other vibrational spectroscopy techniques (e.g. NIR, Raman). In this way the techniques described herein advantageously enable a reliable method to generate calibration models that is unique for a particular type of alcohol to determine its respective branching index.

The disclosed techniques demonstrate that FTIR-ATR spectroscopy may be leveraged to determine the branching index of lightly branched alcohols. For example, the linear calibration model generated from the correlation model may be utilized to estimate the branching index of lightly branched alcohols during synthesis and/or post-synthesis in real-time. In certain embodiments, FTIR-ATR spectrometers may be implemented on-, in- or at-line in a process environment/quality control environment that is associated with the production of lightly branched alcohols. For example, the FTIR-ATR spectrometer may be utilized to determine branching of a lightly branched alcohol. Based on the results of the FTIR-ATR spectra, synthesis conditions and/or operating conditions may be modified to optimize the synthesis of the lightly branched alcohols. In this way, employing FTIR-ATR spectroscopy provides several advantages, such as a cost-effective analysis, eliminating sample preparation, and can be implemented on-, in- or at-line in a process/quality control environment.

### Branching at the 2^{nd} Position

Conventionally, FTIR has been employed to identify (OH) stretching vibrations and quantify hydroxyl group functionalities in alcohol compounds by analyzing spectra bands that reside within 3100 to 3500 cm⁻¹. However, the region between about 1000 to about 1100 cm⁻¹ may provide insight regarding the chemical structure of alcohols, which corresponds to C—C—O stretching vibrations where C—O stretching frequency is coupling to C—C frequencies, demonstrated in Table 12. Without being bound by theory, it is believed that hydrogen bonding, steric effects, and/or electronic effects may affect stretching of the C—C—O band of alcohols. For example, Table 12 demonstrates that the presence of a single branch off the carbon chain at the 2^{nd} position from the hydroxyl group may produce a shift of about 15 cm⁻¹ to lower frequencies from the C—O band position relative to the unbranched parent alcohol. That is, the number of branches (i.e., degree of branching) may shift the wavenumber corresponding to C—C—O vibrations. Accordingly, it is presently recognized that a percentage of molecules that have their first branch at the 2^{nd} position may be determined for the lightly branched alcohols by monitoring the region corresponding to C—C—O vibrations.

| Type of C—C—O Vibration | | Wavenumber (cm⁻¹) |
|---|---|---|
| a. | 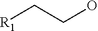 | 1050 |
| b. | 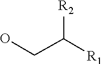 | 1035 |
| c. | 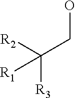 | 1020 |

Table 12 shows band assignments of different types of C—C—O functional groups present in the branched alcohols, as determined via FTIR-ATR spectroscopy.

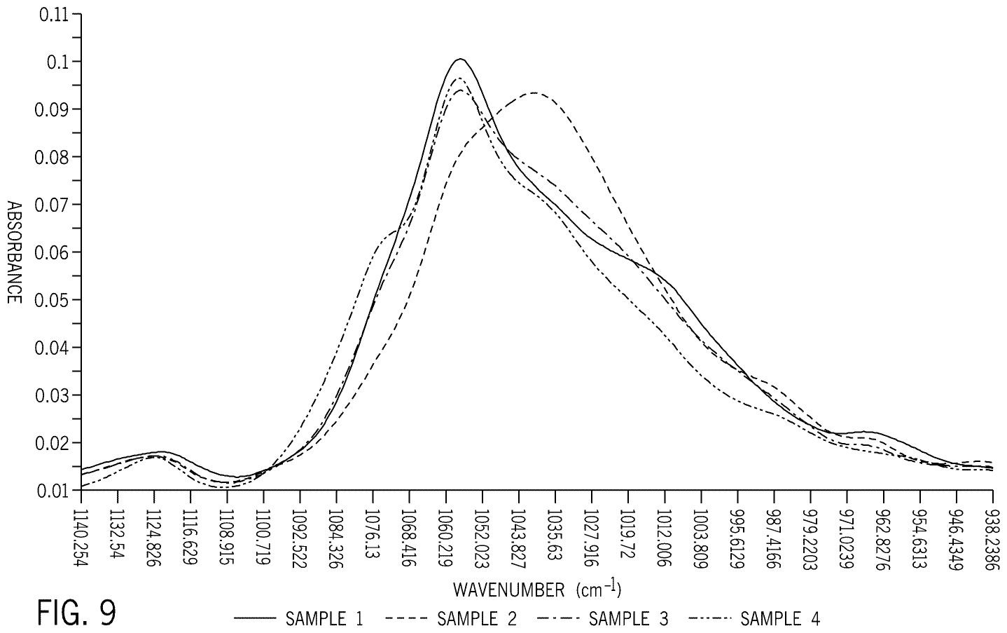

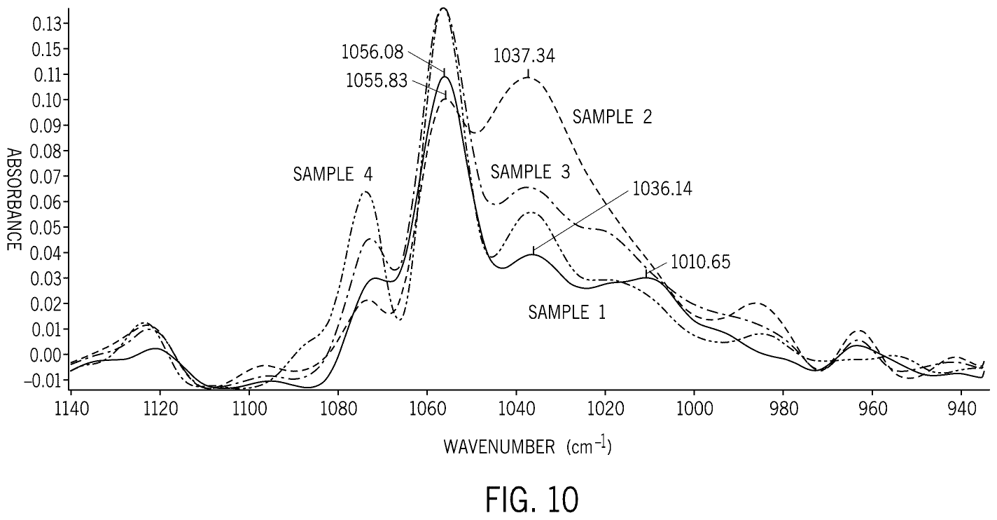

With the foregoing in mind, FIG. 9 is an example FTIR-ATR spectrum of the branched alcohols before signal processing via a Fourier deconvolution (e.g., Fourier self-deconvolution) in the 936 cm⁻¹ to 1140 cm⁻¹ region. The y-axis is representative of absorbance (in reflection mode), and the x-axis is representative of the wavenumbers in cm⁻¹. In the illustrated spectrum, the 936 cm⁻¹ to 1140 cm⁻¹ region includes spectral peaks associated with C—C—O vibrations (as shown in Table 12) and indicates the presence of branching at 2^{nd} position. It should be noted that the while there are spectral differences for each of the samples, such as presence of shoulders for respective peaks, the spectra are not distinguishable. Accordingly, signal processing for curve resolution via Fourier self-deconvolution (FSD) was applied to the data to resolve the various peaks associated with each sample (and branching). In general, FSD employs mathematical algorithms for signal deconvolution to enhance curve resolution while keeping the number of individual profiles to three. FIG. 10 illustrates the results of the deconvolution procedure.

FIG. 10 is the example FTIR-ATR spectrum of the branched alcohols after Fourier self-deconvolution in the in the 936 cm⁻¹ to 1140 cm⁻¹ region. In general, FIG. 10 demonstrates that spectral bands within this region are affected by the presence of branching at the 2^{nd} position. That is, the presence of branching (e.g., one or more branches) at the second position induces an electronic effect, which is validated due to spectral differences between each of the individual samples and is correlated with the structure of the respective alcohol. Due to the observed spectral differences, this spectral region was further utilized to determine the percentage of molecules with their first branch at the second position (i.e., branching at 2^{nd} position). In particular, branching at 2^{nd} position (IR_{Branching at the 2nd position}) was quantified using signal intensity of C—C—O spectral peaks (as demonstrated in Table 12) with the formula (e.g., IR_{Branching at 2nd position} formula):

$${IR}_{Branching at {2}^{nd} position} = \frac{Peak {height}_{R - CH 2 - CH 2 - OH}}{Peak {height}_{R - CH 2 - CH 2 - OH} + Peak {height}_{( R 2 , R 1 ) CH - CH 2 - OH} + Peak {height}_{( R 1 , R 2 , R 3 ) C - CH 2 - OH}} = \frac{{I}_{1050}}{{I}_{1050} + {I}_{1035} + {I}_{1020}}$$

Each peak height is designated as “Iᵥ”, wherein ν is the frequency at a corresponding peak maximum height. In the foregoing formula, the peak height was determined for different types of C—C—O vibration type if applicable. For example, Peak height_{(R—CH2-CH2-OH)} is representative of a peak height that corresponds to 1050 cm⁻¹ (e.g., molecule a in Table 12). To validate the results of the branching at 2^{nd} position as determined by the FTIR-ATR spectra and the preceding formula, a linear calibration model was generated by correlating FTIR-ATR data and branching at 2^{nd} position data acquired via ¹³C NMR spectroscopy, which can be seen in FIG. 11.

With the preceding in mind, FIG. 11 is a graph of a correlation model including FTIR-ATR and ¹³C NMR spectroscopy data. The correlation model includes a linear calibration model (e.g., linear regression model) that was generated using the IR_{Branching at the 2nd position} as determined using the graph from FIG. 10 and the preceding formula and branching at 2^{nd} position data acquired via ¹³C NMR spectroscopy of the branched alcohols. The y-axis represents branching at 2^{nd} position index as measured by FTIR-ATR, while the x-axis percentage of molecules with their first branch in the 2^{nd} position as measured by ¹³C NMR. The linear calibration model demonstrates that there is a good correlation between FTIR-ATR and ¹³C NMR spectroscopy data, which is further demonstrated in Table 13. These results demonstrate that the correlation model (e.g., the linear calibration model) may be utilized to facilitate estimation of a percentage of molecules with their first branch in the 2^{nd} position of branched alcohols using FTIR-ATR spectroscopy. That is, FTIR-ATR data may be acquired of other alcohols at a spectral region ranging from about 936 cm⁻¹ to 1140 cm⁻¹. The acquired spectral data may be inputted into the generated linear regression model to quantify a percentage of molecules with their first branch at 2^{nd} position for the other alcohols. In this way, the present techniques facilitate a fast and reliable way to determine branching properties of lightly branched alcohols.

| | Branching at 2^{nd} |
|---|---|
| | Position Model |
| R² | 0.98 |
| RMSEC (Average number of branches per molecule) | 2.40 |
| RMSEV (Average number of branches per molecule) | 2.93 |

Table 13 shows estimated values of the linear calibration model as shown in FIG. 11. (R²: coefficient of determination, RMSE: root mean square error (C: calibration, V: validation))

The developed modelling approach correlating FTIR and ¹³C NMR data using structural indexes (or chemometrics) may be useful for spectral data of long chain alcohols (e.g., lightly branched C13 alcohols) analyzed by infrared spectroscopy in reflection (or in transmission measurement mode) having their branching at 2^{nd} position index that ranges from 5.1% to 57.6%, as identified by ¹³C NMR. While the methodologies disclosed herein utilize branched C13 alcohols, a similar methodology may be employed to produce a linear calibration model that is specific for long chain alcohols that exhibit greater or lower average branching than the samples described herein or for different types of alcohols (e.g., alcohols greater than or lower than C13), or using other vibrational spectroscopy techniques (e.g. NIR, Raman). In this way the techniques described herein advantageously enable a reliable method to generate calibration models that is unique for a particular type of alcohol to determine a percentage of molecules with their first branch at 2^{nd} position for a respective alcohol. Furthermore, in a generally similar manner as indicated above, the present techniques may be utilized to estimate a percentage of molecules with their first branch at 2^{nd} position for a respective alcohol of lightly branched alcohols during synthesis and/or post-synthesis in real-time. In this way, employing FTIR-ATR spectroscopy provides several advantages, such as a cost-effective analysis, eliminating sample preparation, and can be implemented on-, in- or at-line in a process/quality control environment.

Accordingly, additional aspects of the present disclosure are directed to techniques for determining branching index and percentage of molecules with their first branch at 2^{nd} position for lightly branched alcohols (e.g., LBAs 32) using FTIR-ATR spectroscopy. In particular, the disclosed techniques may leverage the fingerprint region (e.g., wavenumbers ranging 1600 and 600 cm⁻¹) to elucidate behaviors of long chain alcohols to determine branching. For example, the techniques include determining the branching index of the lightly branched alcohols using spectral peaks associated with CH₂ and CH₃ vibrations (e.g., between about 1330 and about 1480 cm⁻¹) and determining percentage of molecules with branching at the 2^{nd} position using spectral peaks associated with C—C—O vibrations (e.g., between about 936 to about 1140 cm⁻¹), respectively. A linear regression model (e.g., correlation model) is generated by correlating FTIR-ATR spectra with NMR spectra (e.g., ¹HMR, ¹³CNMR) to validate and determine branching and branching at the 2^{nd} position of the lightly branched alcohols. The generated models may be subsequently employed to determine branching index and branching at 2^{nd} position of long chain alcohols respectively. The disclosed techniques provide several advantages such as enabling a cost-effective analysis, eliminating sample preparation, and can be implemented on- or at-line in a process/quality control environment (e.g., in a chemical production system).

This written description uses embodiments/examples to disclose the invention, including the best mode, and also to enable any person skilled in the art to practice the invention, including making and using any devices or systems and performing any incorporated methods. The patentable scope of the invention is defined by the claims, and may include other embodiments/examples that occur to those skilled in the art. Such other embodiments/examples are intended to be within the scope of the claims if they have structural elements that do not differ from the literal language of the claims, or if they include equivalent structural elements with insubstantial differences from the literal language of the claims. Many alterations, modifications, and variations will be apparent to those skilled in the art in light of the foregoing description without departing from the spirit or scope of the present disclosure and that when numerical lower limits and numerical upper limits are listed herein, ranges from any lower limit to any upper limit are contemplated.
Embodiment 1. An alcohol composition having one or more branches off of a main carbon chain, wherein the alcohol composition comprises a branching index between about 1.3 and about 1.7 and an average carbon number between about 12.5 and about 13.5 and a kinematics viscosity at 20° C. between 30 to 40 mm²/s.
Embodiment 2. The alcohol composition of embodiment 1, wherein about 10% to about 20% of the alcohol composition has a first branch at position 2 counting from the hydroxyl group.
Embodiment 3. The alcohol composition of any preceding embodiment, wherein about 20% to about 40% of the alcohol has a first branch at position 3 counting from the hydroxyl group.
Embodiment 4. The alcohol composition of any preceding embodiment, wherein about 10% to about 20% of the alcohol has a first branch at position 2 counting from the hydroxyl group, and wherein about 20% to about 40% of the alcohol has a first branch at position 3 counting from the hydroxyl group.
Embodiment 5. The alcohol composition of any preceding embodiment, wherein about 10% to about 20% of the alcohol has a first branch at position 2 counting from the hydroxyl group, wherein about 20% to about 40% of the alcohol has a first branch at position 3 counting from the hydroxyl group, and wherein the alcohol has between about 5% to about 15% first branch at position 4 counting from hydroxyl group.
Embodiment 6. The alcohol composition of any preceding embodiment, wherein about 10% to about 20% of the alcohol has a first branch at position 2 counting from the hydroxyl group, wherein about 20% to about 30% of the alcohol has a first branch at position 3 counting from the hydroxyl group, wherein the alcohol has between about 5% to about 15% first branch at position 4 counting from hydroxyl group, and wherein the alcohol having between about 35% to about 55% first branch at position 5 & 5+ counting from hydroxyl group.
Embodiment 7. The alcohol composition of any preceding embodiment, wherein the alcohol has between about 5% to about 15% first branch at position 4 counting from hydroxyl group.
Embodiment 8. The alcohol composition of any preceding embodiment, wherein the alcohol having between about 35% to about 55% first branch at position 5 & 5+ counting from hydroxyl group.
Embodiment 9. A method, comprising: providing a butene feedstock; generating higher olefins by contacting the butene feedstock in the presence of a catalyst; fractionating the higher olefins to obtain lightly branched olefins; hydroformylating, hydrogenating and fractionating the lightly branched olefins in the presence of a catalyst to produce an alcohol composition, wherein the alcohol composition comprises a branching index between about 1.3 and about 1.7 and an average carbon number between about 12.5 and about 13.5 and a kinematics viscosity at 20° C. between 30 to 40 mm²/s; providing a surfactant precursor; and generating a surfactant by contacting the alcohol composition with the surfactant precursor in the presence of an optional catalyst.
Embodiment 10. The method of embodiment 9, wherein the alcohol composition is used to make nonionic surfactants or anionic surfactants.
Embodiment 11. A surfactant composition, comprising: one or more branched C₁₃ alcohol derived surfactants obtained from a branched C₁₃ alcohol having a branching index between about 1.3 and about 1.7, an average carbon number between about 12.5 and about 13.5 and a kinematics viscosity at 20° C. between 30 to 40 mm²/s; and one or more additives.
Embodiment 12. A detergent formulation comprising the surfactant composition of embodiment 11.
Embodiment 13. The surfactant composition of embodiment 11, wherein the one or more branched C₁₃ alcohol derived surfactants comprises one or more branched C₁₃ alcohol-based sulfates.
Embodiment 14. The surfactant composition of embodiment 11, wherein the one or more branched C₁₃ alcohol derived surfactants comprises one or more branched C₁₃ alcohol derived ethoxylates.
Embodiment 15. The surfactant composition of any preceding embodiment, wherein the one or more additives comprise one or more substantially linear C₁₂-C₁₆ alcohol derived surfactants obtained from a substantially linear C₁₂-C₁₆ alcohol.
Embodiment 16. The surfactant composition of any preceding embodiment, where the substantially linear C₁₂-C₁₆ alcohol has a branching index less than or equal to 0.8.
Embodiment 17. The surfactant composition of any preceding embodiment, wherein the substantially linear C₁₂-C₁₆ alcohol has an average carbon number that is about 12.5 to 15.5.
Embodiment 18. The surfactant composition of any preceding embodiment, wherein a ratio of the one or more branched C₁₃ alcohol derived surfactants to the one or more substantially linear C₁₂-C₁₆ alcohol derived surfactants is greater than 0.5
Embodiment 19. A detergent formulation comprising the surfactant composition of embodiment 15.
Embodiment 20. The surfactant composition of any preceding embodiment, wherein a ratio of the one or more branched C₁₃ alcohol derived surfactants to the one or more substantially linear C₁₂-C₁₆ alcohol derived surfactants is about 0.5:1, 1:1, 2:1, 3:1, 4:1, or 5:1.

1. An alcohol composition having one or more branches off of a main carbon chain, wherein the alcohol composition comprises a branching index between about 1.3 and about 1.7 and an average carbon number between about 12.5 and about 13.5 and a kinematic viscosity at 20° C. between 30 to 40 mm²/s.

2. The alcohol composition of claim 1, wherein about 10% to about 20% of the alcohol composition has a first branch at position 2 counting from the hydroxyl group.

3. The alcohol composition of claim 1, wherein about 20% to about 40% of the alcohol has a first branch at position 3 counting from the hydroxyl group.

4. The alcohol composition of claim 1, wherein about 10% to about 20% of the alcohol has a first branch at position 2 counting from the hydroxyl group, and wherein about 20% to about 40% of the alcohol has a first branch at position 3 counting from the hydroxyl group.

5. The alcohol composition of claim 1, wherein about 10% to about 20% of the alcohol has a first branch at position 2 counting from the hydroxyl group, wherein about 20% to about 40% of the alcohol has a first branch at position 3 counting from the hydroxyl group, and wherein the alcohol has between about 5% to about 15% first branch at position 4 counting from hydroxyl group.

6. The alcohol composition of claim 1, wherein about 10% to about 20% of the alcohol has a first branch at position 2 counting from the hydroxyl group, wherein about 20% to about 30% of the alcohol has a first branch at position 3 counting from the hydroxyl group, wherein the alcohol has between about 5% to about 15% first branch at position 4 counting from hydroxyl group, and wherein the alcohol having between about 35% to about 55% first branch at position 5 & 5+ counting from hydroxyl group.

7. The alcohol composition of claim 1, wherein the alcohol has between about 5% to about 15% first branch at position 4 counting from hydroxyl group.

8. The alcohol composition of claim 1, wherein the alcohol having between about 35% to about 55% first branch at position 5 & 5+ counting from hydroxyl group.

9. A method, comprising:
providing a butene feedstock;
generating higher olefins by contacting the butene feedstock in the presence of a catalyst;
fractionating the higher olefins to obtain lightly branched olefins;
hydroformylating, hydrogenating and fractionating the lightly branched olefins to produce an alcohol composition, wherein the alcohol composition comprises a branching index between about 1.3 and about 1.7 and an average carbon number between about 12.5 and about 13.5 and a kinematic viscosity at 20° C. between 30 to 40 mm²/s;
providing a surfactant precursor; and
generating a surfactant by contacting the alcohol composition with the surfactant precursor in the presence of an optional catalyst.

10. The method of claim 9, wherein the alcohol composition is used to make nonionic surfactants or anionic surfactants.

11. A surfactant composition, comprising:
one or more branched C₁₃ alcohol derived surfactants, obtained from a branched C₁₃ alcohol having a branching index between about 1.3 and about 1.7, an average carbon number between about 12.5 and about 13.5 and a kinematic viscosity at 20° C. between 30 to 40 mm²/s; and
one or more additives.

12. A detergent formulation comprising the surfactant composition of claim 11.

13. The surfactant composition of claim 11, wherein the one or more branched C₁₃ alcohol derived surfactants comprises one or more branched C₁₃ alcohol-based sulfates.

14. The surfactant composition of claim 11, wherein the one or more branched C₁₃ alcohol derived surfactants comprises one or more branched C₁₃ alcohol derived ethoxylates.

15. The surfactant composition of claim 11, wherein the one or more additives comprise one or more substantially linear C₁₂-C₁₆ alcohol derived surfactants, obtained from a substantially linear C₁₂-C₁₆ alcohol.

16. The surfactant composition of claim 15, where the substantially linear C₁₂-C₁₆ alcohol has a branching index less than or equal to 0.8.

17. The surfactant composition of claim 15, wherein the substantially linear C₁₂-C₁₆ alcohol has an average carbon number that is about 12.5 to 15.5.

18. The surfactant composition of claim 15, wherein a ratio of the one or more branched C₁₃ alcohol derived surfactants to the one or more substantially linear C₁₂-C₁₆ alcohol derived surfactants is greater than 0.5.

19. A detergent formulation comprising the surfactant composition of claim 15.

20. The surfactant composition of claim 15, wherein a ratio of the one or more branched C₁₃ alcohol derived surfactants to the one or more substantially linear C₁₂-C₁₆ alcohol derived surfactants is about 0.5:1, 1:1, 2:1, 3:1, 4:1, or 5:1.
